(19) United States
(12) Patent Application Publication
(10) Pub. No.: US 2004/0002115 A1
(43) Pub. Date: Jan. 1, 2004

(54) Method of identifying protein CAMs (constitutively active mutants)

(76) Inventors: Pauline Fraissignes, Marseille (FR); Sabine Gratzer, Grafelfing (DE); Ekkehard Leberer, Germering (DE)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/340,447

(22) Filed: Jan. 10, 2003

Related U.S. Application Data
(60) Provisional application No. 60/360,328, filed on Feb. 27, 2002.

(30) Foreign Application Priority Data
Jan. 12, 2002 (DE) 02000733.2

(51) Int. Cl. G01N 33/53; C12N 9/12; C12N 1/18; C12N 15/74; C07K 14/705

(52) U.S. Cl. 435/7.1; 435/254.2; 435/194; 435/483; 530/350

(57) ABSTRACT

The present invention relates to a method of identifying protein Constitutively Active Mutants (CAMs) and the use thereof.

The present invention relates to a method of identifying protein CAMs and the use thereof.

The G protein-coupled receptors (GPCRs) constitute the largest family of membrane receptors with a common evolutionary origin. They include receptors which respond to environmental ligands (odorants, flavors) or radiations (light of various wavelengths) and to innumerable internal signals (hormones, bioactive amines, neuropeptides, arachidonic acid metabolites, purines, etc. . . . ). This extreme diversity contrasts with their stereotyped structure (seven transmembrane alpha helices, three extracellular loops, three intracellular loops, amino terminus outside and carboxyl terminus inside the cell) and with the limited number of downstream regulatory cascades they control.

The GPCR interacts with an intracellular heterotrimeric G protein consisting of αβγ subunits. Upon binding of the receptor's ligand, the α-subunit dissociates from the β- and γ-subunits, and hydrolyses GTP to GDP. Both Gα and Gβγ can then activate downstream transduction effectors or regulate other receptors (Zwick et al., 1999). in haploid Saccharomyces cerevisiae cells, GPCRs are regulating the mating process. The receptor (Ste2 or Ste3) detects the presence of cells of the opposite mating type (through binding of peptide mating pheromones), and activates intracellular heterotrimeric G proteins, thus initiating the mating process. Gpa1 (α subunit) dissociates from the βγ (Ste4-Ste18) complex which activates downstream elements of the pheromone response pathway which includes a well-characterized mitogen-activated protein kinase (MAP kinase) cascade. The transcription factor Ste12 can then initiate the transcription of several mating factor-inducible genes such as FUS1.

Reports of mammalian GPCRs expressed in yeast indicate that these heterologous proteins can be reliably expressed in yeast and properly inserted into yeast membranes (Tate and Grisshammer, 1996). Reports, e.g. (Price et al., 1995), demonstrate that a large number of heterologous GPCRs interact with the yeast heterotrimeric G protein with sufficient efficacy to induce a growth-promoting signal. In case the GPCR under investigation does not couple to Gpa1, it is co-expressed together with a chimera where the C-terminal part of Gpa1 is replaced by the corresponding amino acids of a given human G_{α} subunit (Brown et al., 2000). A reporter construct (such as pFUS1-HIS3 or pFUS1-lacZ) is then expected to produce a detectable response upon receptor activation.

Increasingly, it is being appreciated that endogenous receptors, and in particular those of the G protein-coupled receptor family, may possess some level of constitutive activity even in the absence of activating mutation. A potentially important physiological ramification of the constitutive activity of such receptors is that the ability of different receptor subtypes (for the same ligand) to spontaneously isomerize to the active state might well differ. Such receptor subtypes would vary substantially in their properties, thus best suiting them for one or another physiological context (Lefkowitz et al., 1993).

The discovery of constitutive GPCR activity presents a theoretical approach to the identification of ligands for orphan receptors. The basic premise for this idea is that different tertiary conformations (i.e. different allosteric change states) of the receptor protein will display different binding domains for ligands, or different binding affinities for the same ligand. Since the mutation of a receptor sequence can only affect the physico-chemical properties of the receptor, but not those of ligands, a change of affinity of a ligand for a receptor ought to be of a similar magnitude for all ligands and not proportional to the ligand's efficacy (Lefkowitz et al., 1993).

The notion that constitutive activation of G protein-coupled receptors could be responsible for hereditary diseases came first from the study of patients suffering of retinis pigmentosa (Robinson et al., 1992). Since then, several other human pathologies have been linked to constitutive activity or aberrant receptors (Dhanasekaran et al., 1995) (Rao and Oprian, 1996) (Duprez et al., 1997) (Jensen et al., 2000). It has now been recognized that very valuable information relevant to treatment of diseases caused by constitutively active receptors (for instance TSH and LH receptors (Spiegel, 1996)) can be directly obtained by identifying compounds which act as inverse agonists to constitutively activated forms of the receptor.

Additionally, some of the therapeutic effects of presently used receptor antagonists may be related to their inverse agonist properties. Recent results (Varma et al., 1999) show that almost all β-adrenergic antagonists (with the exception of pindolol) have inverse agonist properties in the heart of reserpine treated rats.

Down regulation and desensitization of GPCRs is elicited by agonists or as a consequence of spontaneous activity. Thus, inverse agonists upregulate heptahelical receptors by decreasing spontaneous downregulation (Daeffler and Landry, 2000), offering new approaches to tolerance and dependence to drugs. Amino acid residues can be mutated and lead to ligand-independent activation of the receptor and constitutive activation of signaling pathway (Lefkowitz et al., 1993) (Rao and Oprian, 1996) (Sommers et al., 2000) (Konopka et al., 1996) (Alewijnse et al., 2000).

Several techniques have been applied to the discovery or the study of constitutively active receptors. For instance, manipulation of the stoichiometry of receptors and G proteins (mainly over-expression of the receptor) can create a constitutive active receptor system (Chen et al., 2000) (Samama et al., 1997) or site directed mutagenesis on residues such as the highly conserved DRY motif were found to be involved in stabilizing intramolecular interactions (Alewijnse et al., 2000). To date, random mutagenesis has been used in many works to identify CAMs: in a random saturation mutagenesis of a critical region of the Calcium-sensing receptor (Jensen et al., 2000) or in a systematic screening in a mammalian cell based bioassay of a random mutant library of the angiotensin II AT_{1A} receptor (Parnot et al., 2000).

The ease of genetic manipulation of yeast and the availability of an assay that allows detection of a signaling activity made it possible to search through large random mutational libraries to study the spectrum of mutations capable of causing constitutive activation. Ma & al. (Ma et al., 1987) described a fast and reliable method for plasmid construction (Gap Repair) that is based on the efficient repair of a linearized plasmid by recombination with a homologous DNA restriction fragment during yeast transformation.

Additionally, S. cerevisiae does not show such a rapid desensitization process comparable to the ligand-dependent phosphorylation of receptors followed by receptor interaction with arresting, disruption of the interaction receptor-G protein, and in some case sequestration (Tsao et al., 2001). This makes the identification of a constitutive activity much easier.

Previously, a random mutagenesis strategy combined with a yeast based in vivo sub-cloning/screening has been applied successfully on the amino-terminal and transmembrane regions (approximately the first 300 out of 431 residues) of the yeast Ste2 G protein-coupled receptor (Sommers et al., 2000) and on the second intracellular loop of the V2 Vasopressin receptor (Erlenbach et al., 2001). In contrast, the present method allows the systematic identification of activating mutations over the whole open reading frame, without the need of focusing on some regions. Although people usually choose to mutagenise only a part of the coding sequence because they believe that only this region is involved in the mechanism studied (Erlenbach et al., 2001), no doubt would persist and sometimes new prospects on structure-activity would appear.

WO 00/12705 discloses methods for improving the function of heterologous G protein-coupled receptors.

Random mutagenesis on human GPCRs and functionally studied in mammalian cells, were described by (Parnot et al., 2000)) (CAM discovery of Angiotensin II 1A receptor, full length) and (Jensen et al., 2000) (Functional Importance of the Ala¹¹⁶-Pro¹³⁶ Region in the Calcium-sensing Receptor).

Random mutagenesis of a yeast GPCR and functionally studied in yeast cells, in particular CAM discovery of Ste2 (α-factor receptor), random mutagenesis of amino-terminal and transmembrane regions, including Gap Repair were described by (Sommers et al., 2000) and (Sommers and Dumont, 1997)).

Random mutagenesis on a human GPCR functionally studied in yeast cells, in particular coupling properties study of V2 vasopressin receptor, oligonucleotide-directed random mutagenesis of the intracellular loop 2 (228 bp), including Gap repair were described by (Erlenbach et al., 2001)).

CAMs and methods of using them are also disclosed in WO 00/21987. WO 00/06597 discloses endogenous constitutively activated G protein-coupled orphan receptors. WO 00/22129 and WO 00/22131 disclose non-endogenous constitutively activated human G protein-coupled orphan receptors (site directed mutagenesis of GPCRs to generate constitutively activated mutants) and WO 97/21731 an assay for and uses of peptide hormone receptor ligands.

The discovery of constitutively activated mutants (CAMs) is usually the result of a long process of genetic manipulations and assays in mammalian cell culture. Researchers usually choose site directed mutagenesis because of its more straight forward and fast principle (Egan et al., 1998; Alewijnse et al., 2000).

It was a task of the present invention to provide an easy and fast method for identifying CAMs of proteins, e.g. for GPCRs, ion-channel, enzymes.

The present invention provides a method for identifying protein CAMs (constitutively active mutants), wherein

a) a library of mutated sequences of a protein is generated,

b) yeast cells are transformed with such library and

c) the respective protein CAM is identified.

Examples for proteins for which CAMs can be identified are GPCRs, ion-channels, enzymes, e.g. kinases, proteases, transcription factors.

Preferably, protein CAMs of mammalian proteins are identified, e.g. CAMs of human proteins.

The present invention provides a method of identifying protein CAMs (constitutively active mutants) wherein

a) a library of mutated sequences of a protein is generated,

b) yeast cells are co-transformed with the library and a linearized expression vector,

c) the transformed yeast cells are selected for the repair of the plasmid, and

d) protein CAMs are identified by determining the activity of the respective protein mutant.

The present invention provides a method of identifying protein CAMs (constitutively active mutants) wherein

a) a library of mutated sequences of a protein is generated,

b) yeast cells are co-transformed with such library and a linearized expression vector,

c) the transformed yeast cells are selected for the repair of the plasmid, and

d) the previously selected yeast colonies are transferred on a second selective plate where they are screened for the activity of the respective protein mutant.

In a special embodiment of the invention low fidelity PCR is applied on a full length sequences of a particular protein, e.g. a GPCR, preferably a mammalian protein sequence. The PCR products were co-transformed with a linearized expression vector (e.g. containing at each end short sequences homologous to the end of the PCR product) into an engineered yeast strain. The transformed yeast cells were first selected for the repair of the plasmid (e.g. selection by colony forming on a selective medium). The colonies previously selected were replicated an another medium, selective for the activity of the protein, e.g. the receptor (e.g. by the use of a survival reporter gene expressed only upon receptor signaling). Preferably, three or more identical and independent experiments were done to avoid the PCR's bias. The protein CAMs (“mutants”) have an increased basal signaling activity and the same maximum of stimulation than the wild type protein.

A yeast based in vivo discovery of random active mutants can be applied to the entire coding sequence of a protein, e.g. a human receptor. This was done by screening for constitutive mutations of the human sphingosine 1-phosphate receptor EDG5 (Endothelial Differentiation Gene 5) (An et al., 2000) (Hla, 2001).

To obtain a whole set of single point mutants directly (and also to avoid excessive secondary sub-cloning work to find out the activity conferred by each single point mutation), the PCR protocol was optimized to induce an average of less than one point mutation per copy of the gene. Indeed, the high throughput potential of an in vivo subcloning/screening strategy allows us to increase the size of the library without consuming more time/money.

In another embodiment the present invention relates to engineered yeast cells comprising a library of mutants (e.g. GPCR CAMs) and the use of such engineered yeast cells.

For such engineering for example Saccharomyces cerevisiae, Schizosaccharomyces pombe and Candida albicans cells can be used.

The use of such an engineered yeast cell should bring three major improvements at the same time:

screening of a whole library of mutants generated by low fidelity polymerase chain reaction (PCR)

in vivo sub-cloning of each mutated sequences into the expression vector by homologous recombination

in vivo selection of the active mutants using reporter genes

All three in the same engineered yeast cell.

Further advantages are, that the yeast is a powerful tool for the study of mammalian GPCRs and their transduction characteristics because of the high homology between these eukaryotic cells (Price et al., 1995; Hadcock and Pausch, 1999; Botstein et al., 1997); Yeast has a high rate of homologous recombination and the genetic manipulations of yeast are easy (Ma et al., 1987; Oldenburg et al., 1997); Yeast allows in vivo selection of a receptor's activity (Chambers et al., 2000); In vivo screen allows the direct recovery of the plasmid carrying the mutant of interest (CAM) from the micro-organism. Yeast is cheaper to cultivate and engineer than mammalian cells. The technology used to sub-clone and detect the mutants' activity in mammalian cells is far more expensive and qualitative selection and quantification of the mutants' activity can both be done in the same yeast system.

The present method of identifying protein CAMs presents a low cost, fast and powerful method to systematically identify activating mutations along the whole coding sequence of a protein. In contrast to previous work (Parnot et al., 2000), the cloning step is simplified to a simple transformation in yeast and the selection of active mutants is not more than picking growing colonies.

The transposition of the method into mammalian cells confirmed very well the constitutive activity of the mutants screened and selected in yeast. This proves that the method is a suitable alternative to mutant screening in mammalian systems.

Another big advantage of the method is that it immediately discriminates between a moderately active and a highly active mutant (a too high basal activity would not be suitable for agonist discovery, but appropriate for inverse-agonist screening). The growth speed of the colonies on agar selective medium is well correlated to the different “intensities” of constitutive activity observed in a liquid reporter assay.

De-orphaning can also be achieved with this method, e.g. the method can be applied to orphan GPCRs. Therefore, a low fidelity PCR product was co-transfected with the linearized vector into a panel of yeast strains expressing different humanized Gα protein subunits. On selective medium, mutants were selected only from the yeast strain expressing the Gα specific for its coupling. β-Galactosidase detection after growth in a selective medium showed an increased basal activity of the receptor mutant (i.e. an increased expression level of lacZ, controlled by a FUS1 promoter).

The method of identifying protein CAMs, of e.g. GPCR CAMs can be used for:

Identifying agonists; such use is based on the fact that the affinity of a protein CAM, e.g. GPCR CAM for his agonist is increased (Lefkowitz et al., 1993) (MacEwan and Milligan, 1996) (Alewijnse et al., 2000);

Identifying inverse agonists (Chen et al., 2000);

Studying proteins oligomerization, e.g. GPCR oligomerization, depending on their state of activity;

Crystallizing protein, e.g. GPCRs in different tertiary conformations;

De-orphaning: modulators of protein action can be identified with no prior knowledge of the endogenous ligand or protein function.

### FIGURES

FIG. 1: Summary of the Method of Identifying GPCR CAMs.

FIG. 1 summarizes the whole process of the method.

Random mutagenesis of the EDG5 gene was conducted using the yeast CEN expression plasmid p416GPD-Edg5 carrying an URA3 marker (FIG. 2).

FIG. 2: Restriction Map of p416GPD-Edg5 (Nhel)

A Nhel restriction site was created at position 157 bp of the coding sequence of EDG5. Three nucleotides where exchanged by site directed mutagenesis to create the site. This was necessary to conserve, after linearization of the plasmid, only the first 157 bp and the last 101 bp of the open reading frame for homologous recombination.

To allow in vivo recombination, the p416GPD-Edg5 was linearized by double digestion Nhel-Xmal before co-transformation with the low-fidelity PCR amplification product.

FIG. 3: Restriction Map of pcDNA3.1(+)-Edg5

FIG. 4: Solid Phase Assay

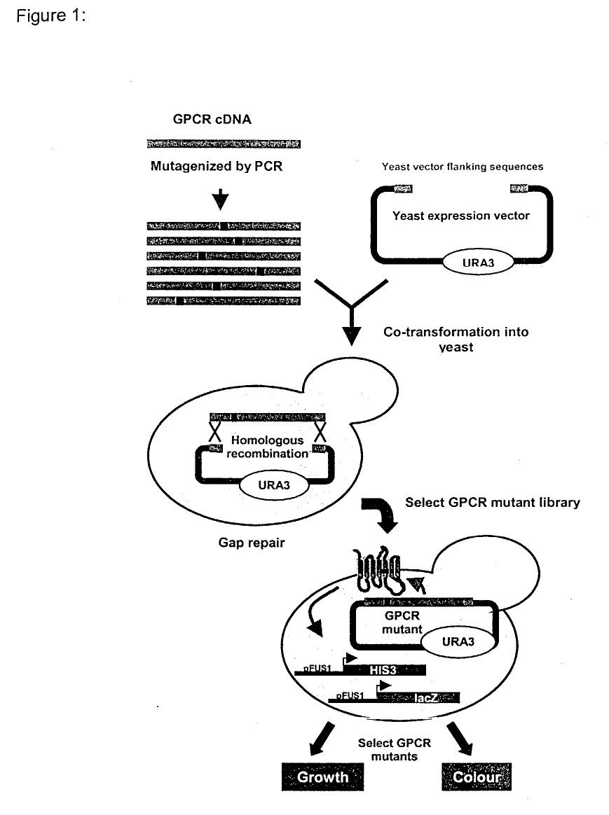

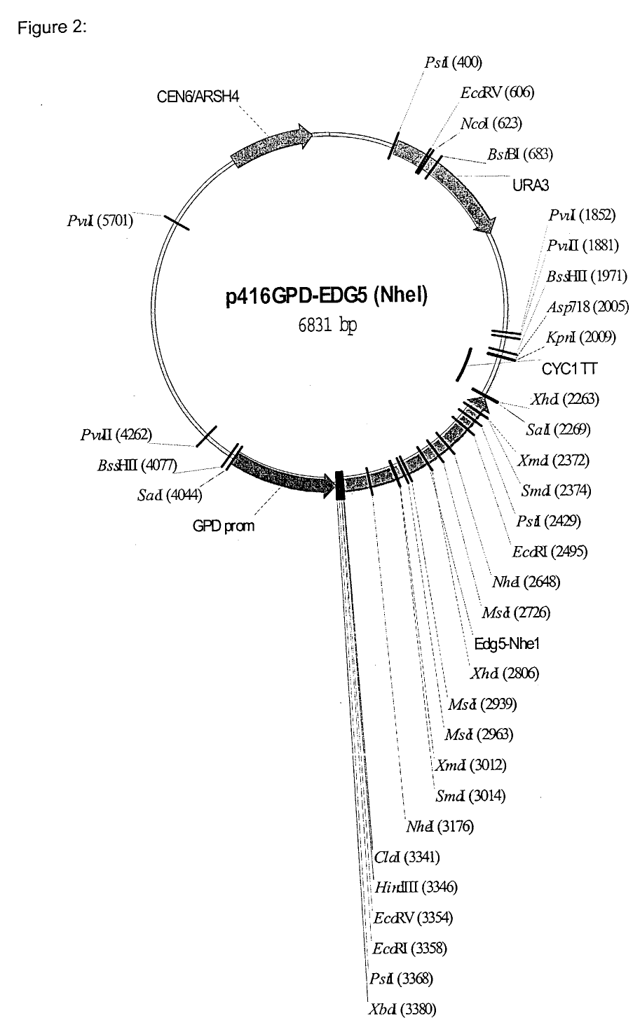

Three colonies of each yeast transformation were tested for the growth and for the β-Galactosidease activity on selective plate:

the first plate (SC Glucose-Ura) shows the normal growth of the colonies;

the second plate (SC Glucose-Ura-His containing 2 mM 3-AT, pH 6.8) shows the growth of the colonies expressing a constitutively activated mutant;

the third plate (SC Glucose-Ura, X-Gal, pH 7) is testing the β-Galactosidase activity (its substrate X-Gal is transformed in a blue metabolite) of the mutants: the frame shows the colored colonies which correspond to the most active clones and correlates with the observations from the second plate.

FIG. 5: Liquid Assay

After 24 hours of growth of the different mutants in selective liquid medium, in the presence of an increasing concentration of Sphingosine 1-Phosphate, β-Galactosidase activity was measured in a calorimetric assay by adding the substrate CPRG, incubating 2 hours and measuring the absorbance at 574 nm.

FIG. 6: Cell Culture Assay

Luciferase activity measured in triplicates after 24 hours of stimulation by a serial dilution of Sphingosine 1-Phosphate.

### EXAMPLES

### Example 1

### Synthesis of the Random Mutational Library

A modified PCR protocol (Svetlov and Cooper, 1998) was used: initial denaturation at 95° C. for 3 min, 30 cycles of denaturation at 95° C. for 5 s, annealing at 50° C. for 5 s, and primer extension at 72° C. for 5 s, and final extension at 72° C. for 5 min, performed on the Cycler PTC-200 (MJ-Research).

The reaction was carried out with 2.5 U of Taq polymerase (Promega) using standard reaction buffer (10 mM Tris-HCl, pH 8.3, 1.5 mM MgCl₂, 50 mM KCl) supplemented with 0.5 mM MnSO₄. An equimolar mix of dNTPs (Amersham Pharmacia Biotech Inc) was used to provide 500 μM of each nucleotide triphosphate in a 100 μl reaction volume. 10 ng of the p416GPD-Edg5 plasmid were used as template. The following oligonucleotides (30 pmol of each) were used as primers for the PCR amplification: EDG5 fwd CAR (SEQ ID NO. 1: 5′-ATG GGC AGC TTG TAC TCG GAG T-3′) and EDG5 rev CAR (SEQ ID NO. 2: 5′-TCA GAA CAC CGT GTT GCC CTC-3′). They correspond exactly to the first 22 and last 21 nucleotides of the receptor's sequence, thus the PCR amplifies exactly the open reading frame.

The PCR product (about 5 μg) was purified by electrophoresis through a 1% agarose-TBE gel followed by elution into 40 μl sterile water (QIAquick Gel Extraction Kit, Qiagen). The DNA final concentration was about 0.1 μg/μl.

### Example 2

### In Vivo Recombination (Gap Repair)

2 μl of the PCR product were cloned into the TA Topo Cloning Vector (Invitrogen) for further qualitative and quantitative analysis of the randomly induced mutations.

The remaining volume of purified PCR product (3-5 μg in 38 μl) was co-transformed with 1 μg of p416GPD-Edg5 (linearized by double digestion with Nhel-Xmal) into about 10⁹ cells of the yeast strain (W303 MATa far1::hisG, sst2::ura3^{FOA}, fus1::HIS3, ΔSte2::Kan^{R}, mfa2-fus1-lacZ::ura3^{FOA}) according to a modified Lithium acetate method (Ito et al., 1983).

The transformed yeast cells were plated on 10 plates SC/Glucose-Ura medium to select for the cells with a “repaired plasmid” (about 10⁸ yeast cells per plate).

After 36 hours incubation at 30° C. (when very small colonies were visible), the transformation plates were replica-plated onto selective medium: SC/Glucose-Ura-His, pH 6.8, containing 2 mM 3-Aminotriazol (3-AT).

### Example 3

### Selection

After 48 hours incubation at 30° C., the colonies still growing were picked and restreaked as patches on a new selective plate (SC/Glucose-Ura-His, pH 6.8, containing 2 mM of 3-AT).

To eliminate false positive mutants (plasmid independent activity), the following steps were performed:

The plasmid from each selected clone was recovered by a Zymolase/SDS treatment protocol adapted from H. Ma & al. (Ma et al., 1987).

After ethanol purification, each plasmid was transformed into E. coli DH5α electro-competent cells. Individual bacterial transformants, one for each mutant, were grown in mini culture for plasmid preparation (QIAprep Spin Miniprep Kit, Qiagen).

The purified DNA was then transformed again into the same yeast strain and each mutant assayed.

### Example 4

### Solid Phase Assay

From a 16 hours culture in SC/glucose-Ura medium, about 3×10⁵ cells of each mutant (in triplicates) were spotted onto three different plates:

SC/Glucose-Ura as a control (to make sure that every spot contains roughly the same number of cells);

SC/Glucose-Ura-His, pH 6.8, containing 2 mM 3-AT;

SC/Glucose-Ura, pH 7, containing 100 μg/ml X-Gal (5-Bromo-4-chloro-3-indolyl β-D-galactopyranoside).

The two first plates were analyzed after 48 hours of growth at 30° C.; the third one was kept for 2 or 3 additional days at 4° C. to develop the blue coloration due to β-Galactosidase activity.

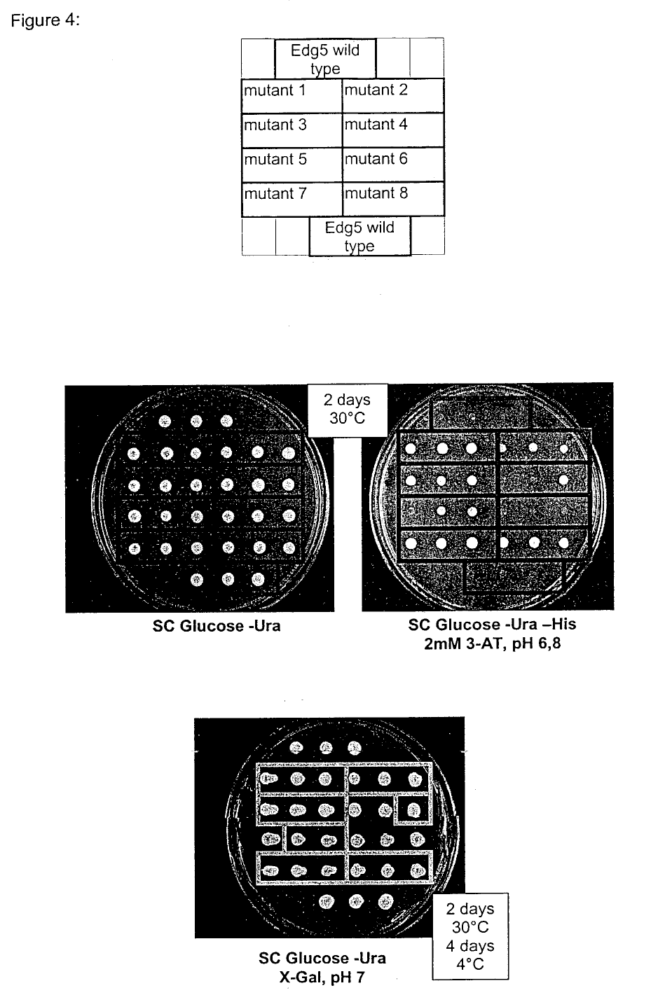

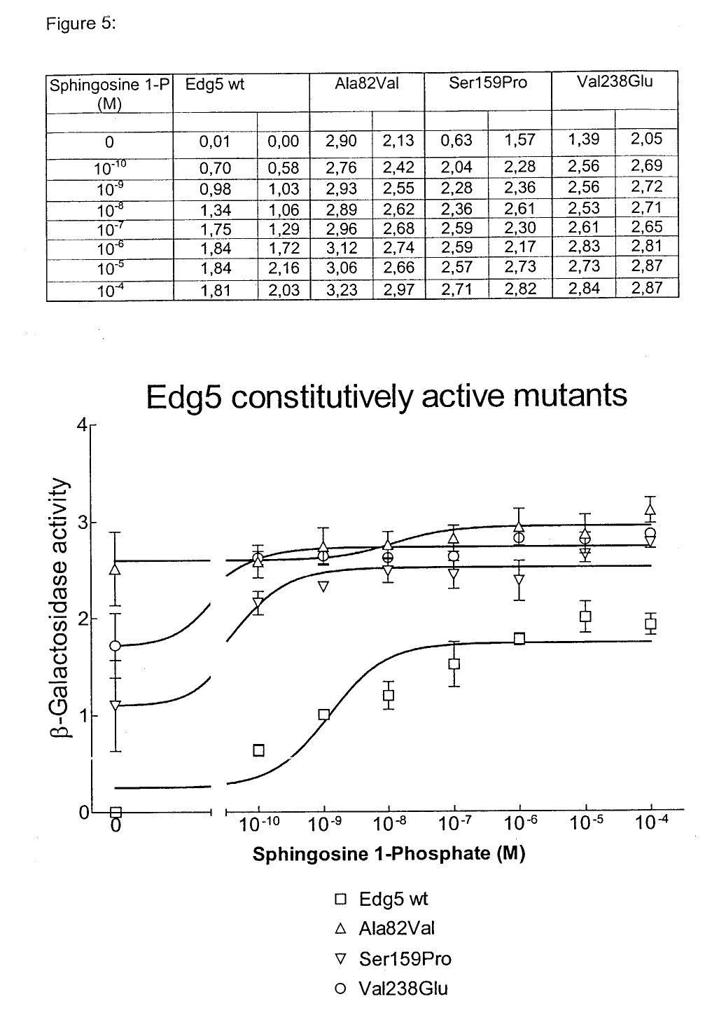

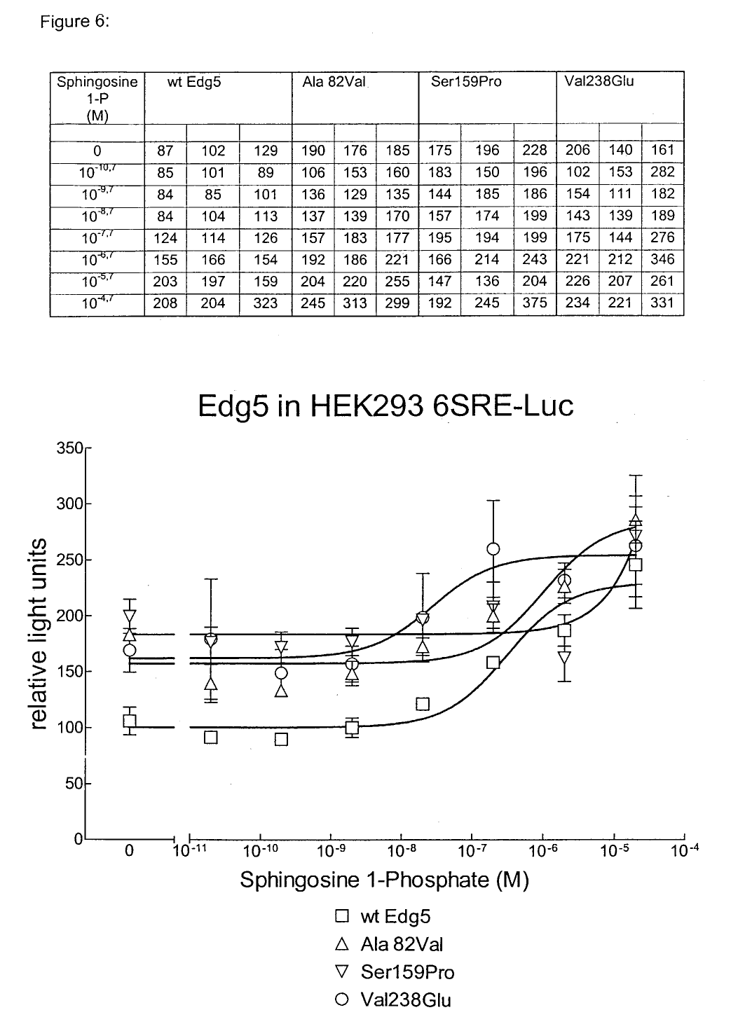

We selected the clones which grew on selective medium (SC/Glucose-Ura-His, pH 6.8, containing 2 mM 3-AT) and gave rise to a blue colored patch on X-Gal medium (SD-Ura, pH 7, containing 100 μg/ml X-Gal). These were candidates for constitutive activity (FIG. 4).

### Example 5

### Liquid Assay in 96 Well Format

The same 16 hours culture was diluted 200 times in selective medium (SC/Glucose-Ura-His, pH 6.8, containing 2 mM 3-AT) and 90 μl were dispensed into the 8 wells of a microtiter plate column already containing 10 μl of a serial dilution of the ligand Spingosine 1-Phosphate (Matreya) (solubilized and diluted in water from 10⁻³ to 10⁻⁹ M).

After 18 to 24 hours of stimulation/growth in a shaking incubator (700 rpm, 30° C.), the β-Galactosidase activity was detected with the substrate Chlorophenolred-β-D-galactopyranoside (CPRG, Boehringer).

### Example 6

### Experimental Procedures in HEK 293

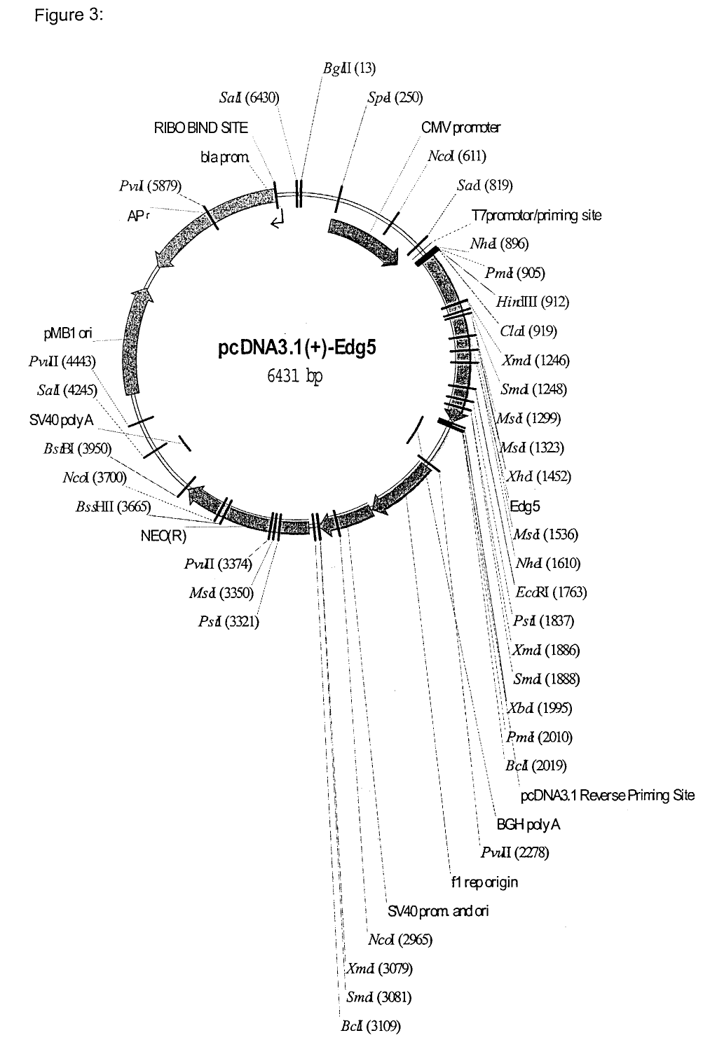

The wild type Edg5 receptor and 3 of the 22 CAMs were sub-cloned into the mammalian expression vector pcDNA3.1(+) to be tested in cell culture (FIG. 3). A HEK 293 cell line stably transfected with the reporter construct 6SRE-Luciferase was utilized for the assay.

This adherent cell line was grown under normal conditions (37° C., 5% CO₂, humid atmosphere) in DMEM-Glutamax (Gibco BRL)+1% Penicillin/Streptomycin+10% Fetal Bovine Serum.

### Example 6.1

### Transfection

Day 1—40.000 cells/well were plated in a white 96-well plate

Day 2—the cells were rinsed with 200 μl Opti-MEM (Gibco BRL) and each well received 100 μl of a transfection mix containing: 0.5 μg receptor plasmid+0.25 μg CMV-β Gal (Promega)+1.2 μl Lipofectamine (Life Technologies) in Opti-MEM.

After 5 hours of incubation with this mix, the wells were emptied and received 180 μl of the normal culture medium (DMEM-Glutamax+1% Penicillin/Streptomycin) containing only 0.5% of Fetal Bovine Serum.

Day 3—20 μl of a 10-fold concentrated serial dilution of Sphingosine 1-Phosphate (from 10⁻⁴ to 10⁻¹⁰ M) was added to each well.

Day 4—the wells were emptied, rinsed with 200 μl phosphate buffer (without calcium and magnesium) and received 50 μl of Glo Lysis Buffer (Promega), after 5 minutes at room temperature, they received 50 μl of Steady-Glo Luciferase Reagent, and the measurement was achieved 5 minutes later in a Luminoskan (Labsystem), 15 seconds integration of the signal.

To normalize the results of the assay, β-Galactosidase activity was measured, from the same plate, after 5 minutes incubation with 25 μl of Gal-Screen Reagent (Tropix), 5 seconds integration of the signal. The luciferase numbers were then divided by the β-Galactosidase numbers.

### Example 7

### Results

### Example 7.1

### TA Cloning Analysis

The analysis of 38 randomly sequenced clones revealed 28 nucleotide mutations: 5 silent mutations, 1 STOP codon and 22 amino acid substitutions. These results suggest that under the experimental conditions the probability for an amino acid substitution to occur in the 354 residues of the wild-type sequence is 0.61.

### Example 7.2

### Analysis of Selected Mutants

The solid phase assay gives a confirmation of the first selection done after replica-plating the gap repair plates on selection plates. After being grown again on selective plate as patches (for confirmation), the plasmid DNA carrying the active mutant was purified, amplified in E. coli and re-transformed into the same yeast strain.

Three colonies of each yeast transformation were tested for growth and for β-Galactosidase activity on selective plates. The FIG. 4 illustrates the clear response from the different mutants obtained in this assay. Here, mutants 1 (contains two mutations, Ala82Val and Ile197Thr) and 7 (Ala82Val only) look the most active (i.e. fast growth on selective plate and blue coloration on X-Gal plate). Mutants 2 (Thr196Ile), 3 (Ser159Pro), 5 (Phe242Leu) and 8 (Ser159Pro and Val215Met) were also selected (at least two of the three clones grew), but appear less active (i.e. the blue coloration is not so obvious). Clone 4 had the same activating Phe242Leu mutation but only one of the three colonies grew. Clone 6 had no activating mutation.

To further characterize the mutants, their activity was tested in a liquid assay. Triplicates of each mutant (re-transformed in yeast) were grown to saturation in a pre-culture. These cell suspensions were diluted 200 times into a medium lacking histidine, permitting the growth of only activated receptors (HIS3 gene under the control of the FUS1 promoter) and distributed in a 96-well microtiterplate together with an increasing concentration of Sphingosine 1-Phosphate. After 24 hours of incubation and shaking at 30° C., the presence of β-Galactosidase activity was measured in a colorimetric assay by adding the substrate CPRG, incubating 2 hours at 30° C. and measuring the absorbance at 574 nm.

FIG. 5 shows that even in the absence of ligand, mutant Ala82Val is hyper-active (which correlates very well with the observations made in the plate assay), while others have a basal activity intermediate between the Ala82Val mutant and the wild type receptor (Ser159Pro and Val238Glu).

Out of three independent screens, 22 mutations have been found to confer constitutive activity to the Edg5 receptor (increased basal activity and the ability to be further stimulated by Sphingosine 1-Phosphate) (Table 1). Interestingly, the mutation Ser159Pro was isolated in each of the three screens, and the mutations Ala82Val and Phe242Leu were isolated in two of the three screens.

### Example 7.3

### HEK 293 Assay

A Serum Responsive Element (SRE)-Luciferase reporter assay in HEK 293 was chosen to verify in mammalian cells the activity of the CAMs selected with the yeast system. A stable HEK 293 cell line carrying the 6SRE-Luciferase construct was transfected with the wild type Edg5 or the mutants Ala82Val, Ser159Pro and Val238Glu. After 24 hours of stimulation, the measurement of Luciferase reflected the receptor's activity.

This assay (FIG. 6) shows an increased basal activity (i.e. in absence of agonist) of the three mutants compared to the wild type, although the maximum response of all four receptors (wild type and mutants) was not changed.

### Example 7.4

### Systematic Screen

To validate a screen, the whole process must be repeated in the same conditions. Indeed, the PCR principle can create an important bias introducing an activating (i.e. the Ala 82->Val mutation found 68 times in the third screen) or inactivating mutation in an early stage of the reaction. This mutation is then present in a high percentage of clones and can mask other interesting point mutations. This has to be circumvented. The best and fastest way would be doing at least three low-fidelity PCRs at the time and all the following steps in parallel.

TABLE 1
Selected mutants analysis
Out of three independent experiments, 22 mutations have been found
to confer constitutive activity to the Edg5 receptor:

| | First | Second | Third | |
|---|---|---|---|---|
| | screen | screen | screen | |
| | (4 active | (3 active | (118 active | |
| | mutants | mutants | mutants | Location |
| Leu 70 -> Pro | | | 2 | Transmembrane |
| | | | | domain 2 |
| Phe 71 -> Leu | | | 7 | Transmembrane |
| | | | | domain 2 |
| Ala 82 -> Val | 2 | | 68 | Transmembrane |
| | | | | domain 2 |
| Val 87 -> Ala | | | 1 | Transmembrane |
| | | | | domain 2 |
| Ser 113 -> Le | | | | Transmembrane |
| | | | | domain 3 |
| Leu 139 -> Pro | | | 2 | Intracellular |
| | | | | loop 2 |
| Ser 155 -> Pro | | | 1 | Transmembrane |
| | | | | domain 4 |
| Ser 159 -> Pro | 1 | 1 | 25 | Transmembrane |
| | | | | domain 4 |
| Val 183 -> Ala | | | 2 | Extracellular |
| | | | | loop 2 |
| Ala 187 -> Thr | | | 1 | Extracellular |
| | | | | loop 2 |
| Lys 188 -> Arg | | | 1 | Extracellular |
| | | | | loop 2 |
| Thr 196 -> Ile | 1 | | | Transmembrane |
| | | | | domain 5 |
| Ile 205 -> Phe | | | 3 | Transmembrane |
| | | | | domain 5 |
| Leu 229 -> Pro | | | 1 | Transmembrane |
| | | | | domain 6 |
| Leu 232 -> Arg | | | 3 | Transmembrane |
| | | | | domain 6 |
| Thr 234 -> Ala | | | 4 | Transmembrane |
| | | | | domain 6 |
| Val 235 -> Ile | | | 4 | Transmembrane |
| | | | | domain 6 |
| Thr 236 -> Ile | | | 1 | Transmembrane |
| | | | | domain 6 |
| Val 238 -> Glu | | | 2 | Transmembrane |
| | | | | domain 6 |
| Val 238 -> Ala | | | 3 | Transmembrane |
| | | | | domain 6 |
| Phe 242 -> Leu | | 2 | 1 | Transmembrane |
| | | | | domain 6 |
| Phe 250 -> Tyr | | | 2 | Transmembrane |
| | | | | domain 6 |

TABLE 2
Nucleotide Sequence of p416 GPD-Edg5

(SEQ ID NO. 3)

| | |
|---|---|
| 1 | TCGCGCGTTT CGGTGATGAC GGTGAAAACC TCTGACACAT GCAGCTCCCG |
| | AGCGCGCAAA GCCACTACTG CCACTTTTGG AGACTGTGTA CGTCGAGGGC |
| 51 | GAGACGGTCA CAGCTTGTCT GTAAGCGGAT GCCGGGAGCA GACAAGCCCG |
| | CTCTGCCAGT GTCGAACAGA CATTCGCCTA CGGCCCTCGT CTGTTCGGGC |
| 101 | TCAGGGCGCG TCAGCGGGTG TTGGCGGGTG TCGGGGCTGG GTTAACTATG |
| | AGTCCCGCGC AGTCGCCCAC AACCGCCCAC AGCCCCGACC GAATTGATAC |
| 151 | CGGCATCAGA GCAGATTGTA CTGAGAGTGC ACCATACCAC AGCTTTTCAA |
| | GCCGTAGTCT CGTCTAACAT GACTCTCACG TCGTATGGTG TCCAAAAGTT |
| 201 | TTCAATTCAT CATTTTTTTT TTATTCTTTT TTTTGATTTC GGTTTCTTTG |
| | AAGTTAAGTA GTAAAAAAAA AATAAGAAAA AAAACTAAAG CCAAAGAAAC |
| 251 | AAATTTTTTT GATTCGGTAA TCTCCGAACA CAAGGAAGAA CGAAGGAAGG |
| | TTTAAAAAAA CTAAGCCATT AGAGGCTTGT CTTCCTTCTT CCTTCCTTCC |
| 301 | AGCACACACT TAGATTGGTA TATATACGCA TATGTAGTGT TGAAGAAACA |
| | TCGTGTCTGA ATCTAACCAT ATATATGCGT ATACATCACA ACTTCTTTGT |
| | PstI |
| | ˜˜˜˜˜˜ |
| 351 | TGAAATTCCC CAGTATTCTT AACCCAACTG CACAGAACAA AAACCTGCAG |
| | ACTTTAACGG GTCATAAGAA TTGGGTTGAC GTGTCTTGTT TTTGGACGTC |
| 401 | GAAACGAAGA TAAATCATGT CGAAAGCTAC ATATAAGGAA CGTGCTGCTA |
| | CTTTGCTTCT ATTTAGTACA GCTTTCGATG TATATTCCTT GCACGACGAT |
| 451 | CTCATCCTAG TCCTGTTGCT GCCAAGCTAT TTAATATCAT GCACGAAAAG |
| | GAGTAGGATC AGGACAACGA CGGTTCGATA AATTATAGTA CGTGCTTTTC |
| 501 | CAAACAAACT TGTGTGCTTC ATTGGATGTT CGTACCACCA AGGAATTACT |
| | GTTTGTTTGA ACACACGAAG TAACCTACAA GCATGGTGGT TCCTTAATGA |
| 551 | GGAGTTAGTT GAAGCATTAG GTCCCAAAAT TTGTTTACTA AAAACACATG |
| | CCTCAATCAA CTTCGTAATC CAGGGTTTTA AACAAATGAT TTTTGTGTAC |
| | EcoRV NcoI |
| | ˜˜˜˜˜˜ ˜˜˜˜˜˜ |
| 601 | TGGATATCTT GACTGATTTT TCCATGGAGG GCACAGTTAA GCCGCTAAAG |
| | ACCTATAGAA CTGACTAAAA AGGTACCTCC CGTGTCAATT CGGCGATTTC |
| | BstBI |
| | ˜˜˜˜˜˜ |
| 651 | GCATTATCCG CCAAGTACAA TTTTTTACTC TTCGAAGACA GAAAATTTGC |
| | CGTAATAGGC GGTTCATGTT AAAAAATGAG AAGCTTCTGT CTTTTAAACG |
| 701 | TGACATTGGT AATACAGTCA AATTGCAGTA CTCTGCGGGT GTATACAGAA |
| | ACTGTAACCA TTATGTCAGT TTAACGTCAT GAGACGCCCA CATATGTCTT |
| 751 | TAGCAGAATG GGCAGACATT ACGAATGCAC ACGGTGTGGT GGGCCCAGGT |
| | ATCGTCTTAC CCGTCTGTAA TGCTTACGTG TGCCACACCA CCCGGGTCCA |
| 801 | ATTGTTAGCG GTTTGAAGCA GGCGGCAGAA GAAGTAACAA AGGAACCTAG |
| | TAACAATCGC CAAACTTCGT CCGCCGTCTT CTTCATTGTT TCCTTGGATC |
| 851 | AGGCCTTTTG ATGTTAGCAG AATTGTCATG CAAGGGCTCC CTATCTACTG |
| | TCCGGAAAAC TACAATCGTC TTAACAGTAC GTTCCCGAGG GATAGATGAC |
| 901 | GAGAATATAC TAAGGGTACT GTTGACATTG CGAAGAGCGA CAAAGATTTT |
| | CTCTTATATG ATTCCCATGA CAACTGTAAC GCTTCTCGCT GTTTCTAAAA |
| 951 | GTTATCGGCT TTATTGCTCA AAGAGACATG GGTGGAAGAG ATGAAGGTTA |
| | CAATAGCCGA AATAACGAGT TTCTCTGTAC CCACCTTCTC TACTTCCAAT |
| 1001 | CGATTGGTTG ATTATGACAC CCGGTGTGGG TTTAGATGAC AAGGGAGACG |
| | GCTAACCAAC TAATACTGTG GGCCACACCC AAATCTACTG TTCCCTCTGC |
| 1051 | CATTGGGTCA ACAGTATAGA ACCGTGGATG ATGTGGTCTC TACAGGATCT |
| | GTAACCCAGT TGTCATATCT TGGCACCTAC TACACCAGAG ATGTCCTAGA |
| 1101 | GACATTATTA TTGTTGGAAG AGGACTATTT GCAAAGGGAA GGGATGCTAA |
| | CTGTAATAAT AACAACCTTC TCCTGATAAA CGTTTCCCTT CCCTACGATT |
| 1151 | GGTAGAGGGT GAACGTTACA GAAAAGCAGG CTGGGAAGCA TATTTGAGAA |
| | CCATCTCCCA CTTGCAATGT CTTTTCGTCC GACCCTTCGT ATAAACTCTT |
| 1201 | GATGCGGCCA GCAAAACTAA AAAACTGTAT TATAAGTAAA TGCATGTATA |
| | CTACGCCGGT CGTTTTGATT TTTTGACATA ATATTCATTT ACGTACATAT |
| 1251 | CTAAACTCAC AAATTAGAGC TTCAATTTAA TTATATCAGT TATTACCCTA |
| | GATTTGAGTG TTTAATCTCG AAGTTAAATT AATATAGTCA ATAATGGGAT |
| 1301 | TGCGGTGTGA AATACCGCAC AGATGCGTAA GGAGAAAATA CCGCATCAGG |
| | ACGCCACACT TTATGGCGTG TCTACGCATT CCTCTTTTAT GGCGTAGTCC |
| 1351 | AAATTGTAAA CGTTAATATT TTGTTAAAAT TCGCGTTAAA TTTTTGTTAA |
| | TTTAACATTT GCAATTATAA AACAATTTTA AGCGCAATTT AAAAACAATT |
| 1401 | ATCAGCTCAT TTTTTAACCA ATAGGCCGAA ATCGGCAAAA TCCCTTATAA |
| | TAGTCGAGTA AAAAATTGGT TATCCGGCTT TAGCCGTTTT AGGGAATATT |
| 1451 | ATCAAAAGAA TAGACCGAGA TAGGGTTGAG TGTTGTTCCA GTTTGGAACA |
| | TAGTTTTCTT ATCTGGCTCT ATCCCAACTC ACAACAAGGT CAAACCTTGT |
| 1501 | AGAGTCCACT ATTAAAGAAC GTGGACTCCA ACGTCAAAGG GCGAAAAACC |
| | TCTCAGGTGA TAATTTCTTG CACCTGAGGT TGCAGTTTCC CGCTTTTTGG |
| 1551 | GTCTATCAGG GCGATGGCCC ACTACGTGAA CCATCACCCT AATCAAGTTT |
| | CAGATAGTCC CGCTACCGGG TGATGCACTT GGTAGTGGGA TTAGTTCAAA |
| 1601 | TTTGGGGTCG AGGTGCCGTA AAGCACTAAA TCGGAACCCT AAAGGGAGCC |
| | AAACCCCAGC TCCACGGCAT TTCGTCATTT AGCCTTGGCA TTTCCCTCGG |
| 1651 | CCCGATTTAG AGCTTGACGG GGAAAGCCGG CGAACGTGGC GAGAAAGGAA |
| | GGGCTAAATC TCGAACTGCC CCTTTCGGCC GCTTGCACCG CTCTTTCCTT |
| 1701 | GGGAAGAAAG CGAAAGGAGC GGGCGCTAGG GCGCTGGCAA GTGTAGCGGT |
| | CCCTTCTTTC GCTTTCCTCG CCCGCGATCC CGCGACCCTT CACATCGCCA |
| 1751 | CACGCTGCGC GTAACCACCA CACCCGCCGC GCTTAATGCG CCGCTACAGG |
| | GTGCGACGCG CATTGGTGGT GTGGGCGGCG CGAATTACGC GGCGATGTCC |
| | ˜˜˜ |
| 1801 | GCGCGTCGCG CCATTCGCCA TTCAGGCTGC GCAACTGTTC GGAAGGCCGA |
| | CGCGCAGCGC GGTAAGCGGT AAGTCCGACG CGTTGACAAC CCTTCCCGCT |
| | PvuI PvuII |
| | ˜˜˜ ˜˜˜˜˜˜ |
| 1851 | TCGGTGCGGG CCTCTTCGCT ATTACGCCAG CTGGCGAAAG GGCGATGTGC |
| | AGCCACGCCC GGAGAAGCGA TAATGCGGTC GACCGCTTTC CCCCTACACG |
| 1901 | TGCAAGGCGA TTAAGTTGGG TAACGCCAGG GTTTTCCCAG TCACGACGTT |
| | ACGTTCCGCT AATTCAACCC ATTGCGGTCC CAAAAGGGTC AGTGCTGCAA |
| | BssHII |
| | ˜˜˜˜˜˜ |
| 1951 | GTAAAACGAC GGCCAGTGAG CGCGCGTAAT ACGACTCACT ATAGGGCGAA |
| | CATTTTGCTG CCGGTCACTC GCGCGCATTA TGCTGAGTGA TATCCCGCTT |
| | KpnI |
| | ˜˜˜˜˜˜ |
| | Asp718 |
| | ˜˜˜˜˜˜ |
| 2001 | TTGGGTACCG GCCGCAAATT AAAGCCTTCG AGCGTCCCAA AACCTTCTCA |
| | AACCCATGGC CGGCGTTTAA TTTCGGAAGC TCGCAGGGTT TTGGAAGAGT |
| 2051 | AGCAAGGTTT TCAGTATAAT GTTACATGCG TACACGCGTC TGTACAGAAA |
| | TCGTTCCAAA AGTCATATTA CAATGTACGC ATGTGCGCAG ACATGTCTTT |
| 2101 | AAAAAGAAAA ATTTGAAATA TAAATAACGT TCTTAATACT AACATAACTA |
| | TTTTTCTTTT TAAACTTTAT ATTTATTGCA AGAATTATGA TTGTATTGAT |
| 2151 | TAAAAAAATA AATACCGACC TAGACTTCAG GTTGTCTAAC TCCTTCCTTT |
| | ATTTTTTTAT TTATCCCTGG ATCTGAAGTC CAACAGATTG AGGAAGGAAA |
| 2201 | TCGGTTAGAG CGGATGTGGG GGGAGGGCGT GAATGTAAGC GTGACATAAC |
| | AGCCAATCTC GCCTACACCC CCCTCCCGCA CTTACATTCG CACTGTATTG |
| | SalI |
| | ˜˜˜˜˜˜˜ |
| | XhoI |
| | ˜˜˜˜˜˜ |
| 2251 | TAATTACATG ACTCGAGGTC GACTCAGAAC ACCGTGTTGC CCTCCAGAAA |
| | ATTAATGTAC TGAGCTCCAG CTGAGTCTTG TGGCACAACG GGAGGTCTTT |
| 2301 | CGTGGGTGAC GTGGGCATGT GCATGCCCCT CTCCAGGGAG CTGGAGCTGC |
| | GCACCCACTG CACCCGTACA CGTACGGGGA GAGGTCCCTC GACCTCGACG |
| | XmaI |
| | ˜˜˜˜˜˜ |
| | SmaI |
| | ˜˜˜˜˜˜ |
| 2351 | GGAGTGGCAG GAGGTGGTGG CCCGGGGTCC CGCCCCGCCT CCGTCCTTGC |
| | CCTCACCGTC CTCCACCACC GGGCCCCAGG GCGGGGCGGA GGCAGGAACG |
| | PstI |
| | ˜˜˜˜˜˜˜ |
| 2401 | ACCCCCACCC CCGGCCGCCA GCACTGCAGC GGCCGAAGCA CCTCCCGCCG |
| | TGGGGGTGGG GGCCGGCGGT CGTGACGTCG CCGGCTTCGT GGAGGGCGGC |
| | EcoRI |
| | ˜˜˜˜˜˜ |
| 2451 | CAGGTCCCGG CTGCGCCACG TGTAGATGAC GGGGTTGAGC AGGGAATTCA |
| | GTCCAGGGCC GACGCGGTGC ACATCTACTG CCCCAACTCG TCCCTTAAGT |
| 2501 | GGGTGGAGAC GGCGAAAAAG TAGTGGGCTT TGTAGAGGAT CGGGCAGGAG |
| | CCCACCTCTG CCGCTTTTTC ATCACCCGAA ACATCTCCTA GCCCGTCCTC |
| 2551 | TGGACGGGAC AGGCATAGTC CAGAAGGAGG ATGCTGAAGG CGGGCAGCCA |
| | ACCTGCCCTG TCCGTATCAG GTCTTCCTCC TACGACTTCC GCCCGTCGGT |
| 2601 | GCAGACGATA AAGACGCCTA GCACGATGGT GACCGTCTTG AGCAGGGCTA |
| | CGTCTGCTAT TTCTGCGGAT CGTGCTACCA CTGGCAGAAC TCGTCCCGAT |
| | NheI |
| | ˜˜ |
| 2651 | GCGTCTGCGG GGCGGCCATG TCAGCGTGGC TTGAGCGGAC CACGCAGTAG |
| | CGCAGACGCC CCGCCGGTAC AGTCGCACCG AACTCGCCTG GTGCGTCATC |
| | MscI |
| | ˜˜˜˜˜˜ |
| 2701 | ATGCGCACGT ACAGGGCCAC GATGGCCAAC AGGATGATGG AGAAGATGGT |
| | TACGCGTGCA TGTCCCGGTG CTACCGGTTG TCCTACTACC TCTTCTACCA |
| 2751 | CACCACGCAC AGCACATAAT GCTTGGCGTA GAGAGGCAGG ACAGTGGAGC |
| | GTGGTGCGTG TCGTGTATTA CGAACCGCAT CTCTCCGTCC TGTCACCTCG |
| | XhoI |
| | ˜˜˜˜˜˜˜ |
| 2801 | AGGCCTCGAG GTGGCCCAGG CAGTTCCAGC CAAGGATGGG CAGGCCACCG |
| | TCCGGAGCTC CACCGGGTCC GTCAAGGTCG GTTCCTACCC GTCCGGTGGC |
| 2851 | AGGACCAGCG AGATGAGCCA CGAGGCCCCG ATGAGCAGAA GCATGCGGCA |
| | TCCTGGTCGC TCTACTCGGT GCTCCGGGGC TACTCGTCTT CGTACGCCGT |
| | MscI |
| | ˜˜˜˜˜˜˜ |
| 2901 | GCTCTTGTCG CTGCCATACA GCTTGACCTT GGCAATGGCC ACGTGGCGCT |
| | CGAGAACAGC GACGGTATGT CGAACTGGAA CCGTTACCGG TGCACCGCGA |
| | MscI |
| | ˜˜˜˜˜˜˜ |
| 2951 | CAATGGCGAT GGCCAGGAGG CTGAAGACAG AGGCCGAGAG CGTGATGAAG |
| | GTTACCGCTA CCGGTCCTCC GACTTCTGTC TCCGGCTCTC GCACTACTTC |
| | XmaI |
| | ˜˜˜˜˜˜ |
| | SmaI |
| | ˜˜˜˜˜˜ |
| 3001 | GCAGAGCCCT CCCGGGCAAA CCACTGCACA GGCGTCAGCC TCAGCGTGAC |
| | CGTCTCGGGA GGGCCCGTTT GGTGACGTGT CCGCAGTCGG AGTCGCACTG |
| 3051 | AGAGCCAGAG AGCAAGGTAT TGGCTACGAA GGCCACGCCT GCCAGTAGAT |
| | TCTCGGTCTC TCGTTCCATA ACCGATGCTT CCGGTGCGGA CGGTCATCTA |
| 3101 | CGGAGGCGGC CAGGTTGCCC AGAAACAGGT ACATTGCCGA GTGGAACTTG |
| | GCCTCCGCCG GTCCAACGGG TCTTTGTCCA TGTAACGGCT CACCTTGAAC |
| | NheI |
| | ˜˜˜˜˜˜˜ |
| 3151 | CTGTTTCGGG CCACCGCAAT GAGCGCTAGC AGGTTTTCCA CCACAATGGC |
| | GACAAAGCCC GGTGGCGTTA CTCGCGATCG TCCAAAAGGT GGTGTTACCG |
| 3201 | GCAACAGAGG ATGACGATGA AGGCCGAGGC CACCTGGCGG GAGGTCGTCT |
| | CGTTGTCTCC TACTGCTACT TCCGGCTCCG GTGGACCGCC CTCCAGCAGA |
| 3251 | CCTGCGTTTC CAGCGTCTCC TTGGTATAAT TATAGTGTTC CTGGACCTTG |
| | GGACGCAAAG GTCGCAGAGG AACCATATTA ATATCACAAG GACCTGGAAC |
| | HindIII |
| | ˜˜˜˜˜˜ |
| | ClaI |
| | ˜˜˜˜˜˜ |
| 3301 | TTGGGGTTCA GGTACTCCGA GTACAAGCTG CCCATTTTAT CGATAAGCTT |
| | AACCCCAAGT CCATGAGGCT CATGTTCGAC GGGTAAAATA GCTATTCGAA |
| | EcoRV PstI |
| | ˜˜˜˜˜˜ ˜˜˜˜˜˜ |
| | EcoRI XbaI |
| | ˜˜˜˜˜˜˜ ˜˜˜˜˜˜ |
| 3351 | GATATCGAAT TCCTGCAGCC CGGCTAGTTC TAGAATCCGT CGAAACTAAG |
| | CTATAGCTTA AGGACGTCGG GCCGATCAAG ATCTTAGGCA GCTTTGATTC |
| 3401 | TTCTGGTGTT TTAAAACTAA AAAAAAGACT AACTATAAAA GTAGAATTTA |
| | AAGACCACAA AATTTTGATT TTTTTTCTGA TTGATATTTT CATCTTAAAT |
| 3451 | AGAAGTTTAA GAAATAGATT TACAGAATTA CAATCAATAC CTACCGTCTT |
| | TCTTCAAATT CTTTATCTAA ATGTCTTAAT GTTAGTTATG GATGCCAGAA |
| 3501 | TATATACTTA TTAGTCAAGT AGGGGAATAA TTTCAGGGAA CTGGTTTCAA |
| | ATATATGAAT AATCAGTTCA TCCCCTTATT AAAGTCCCTT GACCAAAGTT |
| 3551 | CCTTTTTTTT CAGCTTTTTC CAAATCAGAG AGAGCAGAAG GTAATAGAAG |
| | GCAAAAAAAA GTCGAAAAAG GTTTAGTCTC TCTCGTCTTC CATTATCTTC |
| 3601 | GTGTAAGAAA ATGAGATAGA TACATGCGTG GGTCAATTGC CTTGTGTCAT |
| | CACATTCTTT TACTCTATCT ATGTACGCAC CCAGTTAACG CAACACAGTA |
| 3651 | CATTTACTCC AGGCAGGTTG CATCACTCCA TTGAGGTTGT GCCCGTTTTT |
| | GTAAATGAGG TCCGTCCAAC GTAGTGAGGT AACTCCAACA CGGGCAAAAA |
| 3701 | TGCCTGTTTG TGCCCCTGTT CTCTGTAGTT GCGCTAAGAG AATGGACCTA |
| | ACGGACAAAC ACGGGGACAA GAGACATCAA CGCGATTCTC TTACCTGGAT |
| 3751 | TGAACTGATG GTTGGTGAAG AAAACAATAT TTTGGTGCTG GGATTCTTTT |
| | ACTTGACTAC CAACCACTTC TTTTGTTATA AAACCACGAC CCTAAGAAAA |
| 3801 | TTTTTCTGGA TGCCAGCTTA AAAAGCGGGC TCCATTATAT TTAGTGGATG |
| | AAAAAGACCT ACGGTCGAAT TTTTCGCCCG AGGTAATATA AATCACCTAC |
| 3851 | CCAGGAATAA ACTGTTCACC CAGACACCTA CGATGTTATA TATTCTGTGT |
| | GGTCCTTATT TGACAAGTGG GTCTGTGGAT GCTACAATAT ATAAGACACA |
| 3901 | AACCCGCCCC CTATTTTGGG CATGTACGGG TTACAGCAGA ATTAAAAGGC |
| | TTGGGCGGGG GATAAAACCC GTACATGCCC AATGTCGTCT TAATTTTCCG |
| 3951 | TAATTTTTTG ACTAAATAAA GTTAGGAAAA TCACTACTAT TAATTATTTA |
| | ATTAAAAAAC TGATTTATTT CAATCCTTTT AGTGATGATA ATTAATAAAT |
| | SacI |
| | ˜˜˜˜˜˜ |
| 4001 | CGTATTCTTT GAAATGGCAG TATTGATAAT GATAAACTGA GCTCCAGCTT |
| | GCATAAGAAA CTTTACCGTC ATAACTATTA CTATTTGACT CGAGGTCGAA |
| | BssHII |
| | ˜˜˜˜˜˜˜ |
| 4051 | TTGTTCCCTT TAGTGACGGT TAATTGCGCG CTTGGCGTAA TCATGGTCAT |
| | AACAAGGGAA ATCACTCCCA ATTAACGCGC GAACCGCATT AGTACCAGTA |
| 4101 | AGCTGTTTCC TGTGTGAAAT TGTTATCCGC TCACAATTCC ACACAACATA |
| | TCGACAAAGG ACACACTTTA ACAATAGGCG AGTGTTAAGG TGTGTTGTAT |
| 4151 | GGAGCCGGAA GCATAAAGTG TAAAGCCTCC CCTCCCTAAT GAGTGAGGTA |
| | CCTCGGCCTT CGTATTTCAC ATTTCGGACC CCACGGATTA CTCACTCCAT |
| 4201 | ACTCACATTA ATTGCGTTGC GCTCACTGCC CGCTTTCCAG TCGGGAAACC |
| | TGAGTGTAAT TAACGCAACG CGAGTGACGG GCGAAAGGTC AGCCCTTTGG |
| | PvuII |
| | ˜˜˜˜˜˜˜ |
| 4251 | TGTCGTGCCA GCTGCATTAA TGAATCGGCC AACGCGCGGG GAGAGGCGGT |
| | ACAGCACGGT CGACGTAATT ACTTAGCCGG TTGCGCGCCC CTCTCCGCCA |
| 4301 | TTGCGTATTG GGCGCTCTTC CGCTTCCTCG CTCACTGACT CGCTGCGCTC |
| | AACGCATAAC CCGCGAGAAG GCGAAGGAGC GAGTGACTGA GCGACGCGAG |
| 4351 | GGTCGTTCGG CTGCGGCGAG CGGTATCAGC TCACTCAAAG GCGGTAATAC |
| | CCAGCAAGCC GACGCCGCTC GCCATAGTCG AGTGAGTTTC CGCCATTATG |
| 4401 | GGTTATCCAC AGAATCAGGG GATAACGCAG GAAAGAACAT GTGAGCAAAA |
| | CCAATAGGTG TCTTAGTCCC CTATTGCGTC CTTTCTTGTA CACTCGTTTT |
| 4451 | GGCCAGCAAA AGGCCAGGAA CCGTAAAAAG GCCGCGTTGC TGGCGTTTTT |
| | CCGGTCGTTT TCCGGTCCTT GGCATTTTTC CGGCGCAACG ACCGCAAAAA |
| 4501 | CCATAGGCTC CGCCCCCCTG ACGAGCATCA CAAAAATCGA CGCTCAAGTC |
| | GGTATCCGAG GCGGGGGGAC TGCTCGTAGT GTTTTTAGCT GCGAGTTCAG |
| 4551 | AGAGGTGGCG AAACCCGACA GGACTATAAA GATACCAGGC GTTTCCCCCT |
| | TCTCCACCGC TTTGGGCTGT CCTGATATTT CTATGGTCCG CAAAGGGGGA |
| 4601 | GGAAGCTCCC TCGTGCGCTC TCCTGTTCCG ACCCTGCCGC TTACCGGATA |
| | CCTTCGAGGG AGCACGCGAG AGGACAAGGC TGGGACGGCG AATGGCCTAT |
| 4651 | CCTGTCCGCC TTTCTCCCTT CGGGAAGCGT GGCGCTTTCT CATAGCTCAC |
| | GGACAGGCGG AAAGAGGGAA GCCCTTCGCA CCGCGAAAGA GTATCGAGTG |
| 4701 | GCTGTAGGTA TCTCAGTTCG GTGTAGGTCG TTCGCTCCAA GCTGGGCTGT |
| | CGACATCCAT AGAGTCAAGC CACATCCAGC AAGCGAGGTT CGACCCGACA |
| 4751 | GTGCACGAAC CCCCCGTTCA GCCCGACCGC TGCGCCTTAT CCGGTAACTA |
| | CACGTGCTTG GGGGGCAAGT CGGGCTGGCG ACGCGGAATA GGCCATTGAT |
| 4801 | TCGTCTTGAG TCCAACCCGG TAAGACACGA CTTATCGCCA CTCGCAGCAG |
| | AGCAGAACTC AGGTTGGGCC ATTCTGTGCT GAATAGCGGT GACCGTCGTC |
| 4851 | CCACTGGTAA CAGGATTAGC AGAGCGAGGT ATGTAGGCGG TGCTACAGAG |
| | GGTGACCATT GTCCTAATCG TCTCGCTCCA TACATCCGCC ACGATGTCTC |
| 4901 | TTCTTGAAGT GGTGGCCTAA CTACGGCTAC ACTAGAAGGA CAGTATTTCC |
| | AAGAACTTCA CCACCGGATT GATGCCGATG TGATCTTCCT GTCATAAACC |
| 4951 | TATCTGCGCT CTGCTGAAGC CAGTTACCTT CGGAAAAAGA GTTGGTAGCT |
| | ATAGACGCGA GACGACTTCG GTCAATGGAA GCCTTTTTCT CAACCATCGA |
| 5001 | CTTGATCCGG CAAACAAACC ACCGCTGGTA GCGGTGGTTT TTTTGTTTGC |
| | GAACTAGGCC GTTTGTTTGG TGGCGACCAT CGCCACCAAA AAAACAAACG |
| 5051 | AAGCAGCAGA TTACGCGCAG AAAAAAAGCA TCTCAAGAAG ATCCTTTGAT |
| | TTCGTCGTCT AATGCGCGTC TTTTTTTCCT AGAGTTCTTC TAGGAAACTA |
| 5101 | CTTTTCTACG GGGTCTGACG CTCAGTGGAA CGAAAACTCA CGTTAAGGGA |
| | GAAAAGATGC CCCAGACTGC GAGTCACCTT GCTTTTGAGT GCAATTCCCT |
| 5151 | TTTTGGTCAT GAGATTATCA AAAAGGATCT TCACCTAGAT CCTTTTAAAT |
| | AAAACCAGTA CTCTAATAGT TTTTCCTAGA AGTGGATCTA GGAAAATTTA |
| 5201 | TAAAAATGAA GTTTTAAATC AATCTAAAGT ATATATGAGT AAACTTGGTC |
| | ATTTTTACTT CAAAATTTAG TTAGATTTCA TATATACTCA TTTGAACCAG |
| 5251 | TGACAGTTAC CAATGCTTAA TCAGTGAGGC ACCTATCTCA GCGATCTGTC |
| | ACTGTCAATG GTTACGAATT AGTCACTCCG TGGATAGAGT CGCTAGACAG |
| 5301 | TATTTCGTTC ATCCATAGTT GCCTGACTCC CCGTCGTGTA GATAACTACG |
| | ATAAAGCAAG TAGGTATCAA CGGACTGAGG GGCAGCACAT CTATTGATGC |
| 5351 | ATACGGGAGG GCTTACCATC TGGCCCCAGT GCTGCAATGA TACCGCGAGA |
| | TATGCCCTCC CGAATGGTAG ACCGGGGTCA CGACGTTACT ATGGCGCTCT |
| 5401 | CCCACGCTCA CCGGCTCCAG ATTTATCAGC AATAAACCAG CCAGCCGGAA |
| | GGGTGCGAGT GGCCGAGGTC TAAATAGTCG TTATTTGGTC GGTCGGCCTT |
| 5451 | GGGCCGAGCG CAGAAGTGGT CCTGCAACTT TATCCGCCTC CATCCAGTCT |
| | CCCGGCTCGC GTCTTCACCA GGACGTTGAA ATAGGCGGAG GTAGGTCAGA |
| 5501 | ATTAATTGTT GCCGGGAAGC TAGAGTAAGT AGTTCGCCAG TTAATAGTTT |
| | TAATTAACAA CGGCCCTTCG ATCTCATTCA TCAAGCGGTC AATTATCAAA |
| 5551 | GCGCAACGTT GTTGCCATTG CTACAGGCAT CGTGGTGTCA CGCTCGTCGT |
| | CGCGTTGCAA CAACGGTAAC GATGTCCGTA GCACCACAGT GCGAGCAGCA |
| 5601 | TTGGTATGGC TTCATTCAGC TCCGGTTCCC AACGATCAAG GCGAGTTACA |
| | AACCATACCG AAGTAAGTCG AGGCCAAGGG TTGCTAGTTC CGCTCAATGT |
| | PvuI |
| | ˜˜˜˜ |
| 5651 | TGATCCCCCA TGTTGTGCAA AAAAGCGGTT AGCTCCTTCG GTCCTCCGAT |
| | ACTAGGGGGT ACAACACGTT TTTTCGCCAA TCGAGGAAGC CAGGAGGCTA |
| | PvuI |
| | ˜˜ |
| 5701 | CGTTGTCAGA AGTAAGTTGG CCGCAGTGTT ATCACTCATG GTTATGGCAG |
| | GCAACAGTCT TCATTCAACC GGCGTCACAA TAGTGAGTAC CAATACCGTC |
| 5751 | CACTGCATAA TTCTCTTACT GTCATGCCAT CCGTAAGATG CTTTTCTGTG |
| | GTGACGTATT AAGAGAATGA CAGTACGGTA GGCATTCTAC GAAAAGACAC |
| 5801 | ACTGGTGAGT ACTCAACCAA GTCATTCTGA GAATAGTGTA TGCGGCGACC |
| | TGACCACTCA TGAGTTGGTT CAGTAAGACT CTTATCACAT ACGCCGCTGG |
| 5851 | GAGTTGCTCT TGCCCGGCGT CAATACGGGA TAATACCGCG CCACATAGCA |
| | CTCAACGAGA ACGGGCCGCA GTTATGCCCT ATTATGGCGC GGTGTATCGT |
| 5901 | GAACTTTAAA AGTGCTCATC ATTGGAAAAC GTTCTTCGGG GCGAAAACTC |
| | CTTGAAATTT TCACGAGTAG TAACCTTTTG CAAGAAGCCC CGCTTTTGAG |
| 5951 | TCAAGGATCT TACCGCTGTT GAGATCCAGT TCGATGTAAC CCACTCGTGC |
| | AGTTCCTAGA ATGGCGACAA CTCTAGGTCA AGCTACATTG GGTGAGCACG |
| 6001 | ACCCAACTGA TCTTCAGCAT CTTTTACTTT CACCAGCGTT TCTGGGTGAG |
| | TGGGTTGACT AGAAGTCGTA GAAAATGAAA GTGGTCGCAA AGACCCACTC |
| 6051 | CAAAAACAGG AAGGCAAAAT GCCGCAAAAA AGGGAATAAG GGCGACACGG |
| | GTTTTTGTCC TTCCGTTTTA CGGCGTTTTT TCCCTTATTC CCGCTGTGCC |
| 6101 | AAATGTTGAA TACTCATACT CTTCCTTTTT CAATATTATT GAAGCATTTA |
| | TTTACAACTT ATGAGTATGA GAAGGAAAAA GTTATAATAA CTTCGTAAAT |
| 6151 | TCAGGGTTAT TGTCTCATGA GCGGATACAT ATTTGAATGT ATTTAGAAAA |
| | AGTCCCAATA ACAGAGTACT CGCCTATGTA TAAACTTACA TAAATCTTTT |
| 6201 | ATAAACAAAT AGGGGTTCCG CGCACATTTC CCCGAAAAGT GCCACCTGGG |
| | TATTTGTTTA TCCCCAAGGC GCGTGTAAAG GGGCTTTTCA CGGTGGACCC |
| 6251 | TCCTTTTCAT CACGTGCTAT AAAAATAATT ATAATTTAAA TTTTTTAATA |
| | AGGAAAAGTA GTGCACGATA TTTTTATTAA TATTAAATTT AAAAAATTAT |
| 6301 | TAAATATATA AATTAAAAAT AGAAAGTAAA AAAAGAAATT AAAGAAAAAA |
| | ATTTATATAT TTAATTTTTA TCTTTCATTT TTTTCTTTAA TTTCTTTTTT |
| 6351 | TAGTTTTTGT TTTCCGAAGA TGTAAAAGAC TCTAGGGGGA TCGCCAACAA |
| | ATCAAAAACA AAAGGCTTCT ACATTTTCTG AGATCCCCCT AGCGGTTGTT |
| 6401 | ATACTACCTT TTATCTTGCT CTTCCTGCTC TCAGGTATTA ATGCCGAATT |
| | TATGATGGAA AATAGAACGA GAAGGACGAG AGTCCATAAT TACGGCTTAA |
| 6451 | GTTTCATCTT GTCTGTGTAG AAGACCACAC ACGAAAATCC TGTGATTTTA |
| | CAAAGTAGAA CAGACACATC TTCTGGTGTG TGCTTTTAGG ACACTAAAAT |
| 6501 | CATTTTACTT ATCGTTAATC GAATGTATAT CTATTTAATC TGCTTTTCTT |
| | GTAAAATGAA TAGCAATTAG CTTACATATA GATAAATTAG ACGAAAAGAA |
| 6551 | GTCTAATAAA TATATATGTA AAGTACGCTT TTTGTTGAAA TTTTTTAAAC |
| | CAGATTATTT ATATATACAT TTCATGCGATA AAACAACTTT AAAAAATTTG |
| 6601 | CTTTGTTTAT TTTTTTTTCT TCATTCCGTA ACTCTTCTAC CTTCTTTATT |
| | GAAACAAATA AAAAAAAAGA AGTAAGGCAT TGAGAAGATG GAAGAAATAA |
| 6651 | TACTTTCTAA AATCCAAATA CAAAACATAA AAATAAATAA ACACAGAGTA |
| | ATGAAAGATT TTAGGTTTAT GTTTTGTATT TTTATTTATT TGTGTCTCAT |
| 6701 | AATTCCCAAA TTATTCCATC ATTAAAAGAT ACGAGGCGCG TGTAAGTTAC |
| | TTAAGGGTTT AATAAGGTAG TAATTTTCTA TGCTCCGCGC ACATTCAATG |
| 6751 | AGGCAAGCGA TCCGTCCTAA GAAACCATTA TTATCATGAC ATTAACCTAT |
| | TCCGTTCGCT AGGCAGGATT CTTTGGTAAT AATAGTACTG TAATTGGATA |
| 6801 | AAAAATAGGC GTATCACGAG GCCCTTTCGT C |
| | TTTTTATCCG CATAGTGCTC CGGGAAAGCA G |

TABLE 3
Nucleotide Sequence of pcDNA3.1 (+)-Edg 5

(SEQ ID NO. 4)

| | |
|---|---|
| SalI | BglII |
| | ˜˜˜ ˜˜˜˜˜˜ |
| 1 | GACGGATCGG GAGATCTCCC GATCCCCTAT GGTGCACTCT CAGTACAATC |
| | CTGCCTAGCC CTCTAGAGGG CTAGGGGATA CCACGTGAGA GTCATGTTAG |
| 51 | TGCTCTGATG CCGCATAGTT AAGCCAGTAT CTGCTCCCTG CTTGTGTGTT |
| | ACGAGACTAC GGCGTATCAA TTCGGTCATA GACGAGGGAC GAACACACAA |
| 101 | GGAGGTCGCT GAGTAGTGCG CGAGCAAAAT TTAAGCTACA ACAAGGCAAG |
| | CCTCCAGCGA CTCATCACGC GCTCGTTTTA AATTCGATGT TGTTCCGTTC |
| 151 | GCTTGACCGA CAATTGCATG AAGAATCTGC TTAGGGTTAG GCGTTTTGCG |
| | CGAACTGGCT GTTAACGTAC TTCTTAGACG AATCCCAATC CGCAAAACGC |
| | SpeI |
| | ˜˜˜˜ |
| 201 | CTGCTTCGCG ATGTACGGGC CAGATATACG CGTTGACATT GATTATTGAC |
| | GACGAAGCGC TACATGCCCG GTCTATATGC GCAACTGTAA CTAATAACTG |
| | SpeI |
| | ˜˜˜˜ |
| 251 | TAGTTATTAA TAGTAATCAA TTACGGGGTC ATTAGTTCAT AGCCCATATA |
| | ATCAATAATT ATCATTAGTT AATGCCCCAG TAATCAAGTA TCGGGTATAT |
| 301 | TGGAGTTCCG CGTTACATAA CTTACGGTAA ATGGCCCGCC TGGCTGACCG |
| | ACCTCAAGGC GCAATGTATT GAATGCCATT TACCGGGCGG ACCGACTGGC |
| 351 | CCCAACGACC CCCGCCCATT GACGTCAATA ATGACGTATG TTCCCATAGT |
| | GGGTTGCTGG GGGCGGGTAA CTGCAGTTAT TACTGCATAC AAGGGTATCA |
| 401 | AACGCCAATA GGGACTTTCC ATTGACGTCA ATGGGTGGAG TATTTACGGT |
| | TTGCGGTTAT CCCTGAAAGG TAACTGCAGT TACCcACCTC ATAAATGCCA |
| 451 | AAACTGCCCA CTTGGCAGTA CATCAAGTGT ATCATATGCC AAGTACGCCC |
| | TTTGACGGGT GAACCGTCAT GTAGTTCACA TAGTATACGG TTCATGCGGG |
| 501 | CCTATTGACG TCAATGACGG TAAATGGCCC GCCTGGCATT ATGCCCAGTA |
| | GGATAACTGC AGTTACTGCC ATTTACCGGG CGGACCGTAA TACGGGTCAT |
| 551 | CATGACCTTA TGGGACTTTC CTACTTGGCA GTACATCTAC GTATTAGTCA |
| | GTACTGGAAT ACCCTGAAAG GATGAACCGT CATGTAGATG CATAATCAGT |
| | NcoI |
| | ˜˜˜˜˜˜˜ |
| 601 | TCGCTATTAC CATGGTGATG CGGTTTTGGC AGTACATCAA TGGGCGTGGA |
| | AGCGATAATG GTACCACTAC GCCAAAACCG TCATGTAGTT ACCCGCACCT |
| 651 | TAGCGGTTTG ACTCACGGGG ATTTCCAAGT CTCCACCCCA TTGACGTCAA |
| | ATCGCCAAAC TGAGTGCCCC TAAAGGTTCA GAGGTGGGGT AACTGCAGTT |
| 701 | TGGGAGTTTG TTTTGGCACC AAAATCAACG GGACTTTCCA AAATGTCGTA |
| | ACCCTCAAAC AAAACCGTGG TTTTAGTTGC CCTGAAAGGT TTTACAGCAT |
| 751 | ACAACTCCGC CCCATTGACG CAAATGGGCG GTAGGCGTGT ACGGTGGGAG |
| | TGTTGAGGCG GGGTAACTGC GTTTACCCGC CATCCGCACA TGCCACCCTC |
| | SacI |
| | ˜˜˜˜˜˜˜ |
| 801 | GTCTATATAA GCAGAGCTCT CTGGCTAACT AGAGAACCCA CTGCTTACTG |
| | CAGATATATT CGTCTCGAGA GACCGATTGA TCTCTTGGGT GACGAATGAC |
| | NheI |
| | ˜˜˜˜˜˜ |
| 851 | GCTTATCGAA ATTAATACGA CTCACTATAG GGAGACCCAA GCTGGCTAGC |
| | CGAATAGCTT TAATTATGCT GAGTGATATC CCTCTGGGTT CGACCGATCG |
| | HindIII |
| | ˜˜˜˜˜˜ |
| | PmeI ClaI |
| | ˜˜˜˜˜˜˜˜ ˜˜˜˜˜˜˜ |
| 901 | GTTTAAACTT AAGCTTATCG ATAAAATGGG CAGCTTGTAC TCGGAGTACC |
| | CAAATTTGAA TTCGAATAGC TATTTTACCC GTCGAACATG AGCCTCATGG |
| 951 | TGAACCCCAA CAAGGTCCAG GAACACTATA ATTATACCAA GGAGACGCTG |
| | ACTTGGGGTT GTTCCAGGTC CTTGTGATAT TAATATGGTT CCTCTGCGAC |
| 1001 | GAAACGCAGG AGACGACCTC CCGCCAGGTG GCCTCGGCCT TCATCGTCAT |
| | CTTTGCGTCC TCTGCTGGAG GGCGGTCCAC CGGAGCCGGA AGTAGCAGTA |
| 1051 | CCTCTGTTGC GCCATTGTGG TGGAAAACCT TCTGGTGCTC ATTGCGGTGG |
| | GGAGACAACG CGGTAACACC ACCTTTTGGA AGACCACGAG TAACGCCACC |
| 1101 | CCCGAAACAG CAAGTTCCAC TCGGCAATGT ACCTGTTTCT GGGCAACCTG |
| | GGGCTTTGTC GTTCAAGGTG AGCCGTTACA TGGACAAAGA CCCGTTGGAC |
| 1151 | GCCGCCTCCG ATCTACTGGC AGGCGTGGCC TTCGTAGCCA ATACCTTGCT |
| | CGGCGGAGGC TAGATGACCG TCCGCACCGG AAGCATCGGT TATGGAACGA |
| | XmaI |
| | ˜˜˜˜˜˜SmaI |
| 1201 | CTCTGGCTCT GTCACGCTGA GGCTGACGCC TGTGCAGTGG TTTGCCCGGG |
| | GAGACCGAGA CAGTGCGACT CCGACTGCGG ACACGTCACC AAACGGGCCC |
| | MscI |
| | ˜˜˜˜˜ |
| 1251 | AGGGCTCTGC CTTCATCACG CTCTCGGCCT CTGTCTTCAG CCTCCTGGCC |
| | TCCCGAGACC GAAGTAGTGC GAGAGCCGGA GACAGAAGTC GGAGGACCGG |
| | MscI MscI |
| | ˜ ˜˜˜˜˜˜ |
| 1301 | ATCGCCATTG AGCGCCACGT GGCCATTGCC AAGGTCAAGC TGTATGGCAG |
| | TAGCGGTAAC TCGCGGTGCA CCGGTAACGG TTCCAGTTCG ACATACCGTC |
| 1351 | CGACAAGAGC TGCCGCATGC TTCTGCTCAT CGGGGCCTCG TGGCTCATCT |
| 2351 | AGCGTGACCG CTACACTTGC CAGCGCCCTA GCGCCCGCTC CTTTCGCTTT |
| | TCGCACTGGC GATGTGAACG GTCGCGGGAT CGCGCGCGAG GAAAGCGAAA |
| 2401 | CTTCCCTTCC TTTCTCGCCA CGTTCGCCOG CTTTCCCCGT CAAGCTCTAA |
| | GAAGGGAAGG AAAGAGCGGT GCAAGCGGCC GAAAGGGGCA GTTCGAGATT |
| 2451 | ATCGGGGGCT CCCTTTAGGG TTCCGATTTA GTGCTTTACG GCACCTCGAC |
| | TAGCCCCCGA GGGAAATCCC AAGGCTAAAT CACGAAATGC CGTGGAGCTG |
| 2501 | CCCAAAAAAC TTGATTAGGG TGATGGTTCA CGTAGTGGGC CATCGCCCTG |
| | GGGTTTTTTG AACTAATCCC ACTACCAAGT GCATCACCCG GTAGCGGGAC |
| 2551 | ATAGACGGTT TTTCGCCCTT TGACGTTGGA GTCCACGTTC TTTAATAGTG |
| | TATCTGCCAA AAAGCGGGAA ACTGCAACCT CAGGTGCAAG AAATTATCAC |
| 2601 | GACTCTTCTT CCAAACTGGA ACAACACTCA ACCCTATCTC GGTCTATTCT |
| | CTGAGAACAA GGTTTGACCT TGTTGTGAGT TGGGATAGAG CCAGATAAGA |
| 2651 | TTTGATTTAT AAGGGATTTT GCCGATTTCG GCCTATTGGT TAAAAAATGA |
| | AAACTAAATA TTCCCTAAAA CGGCTAAAGC CGGATAACCA ATTTTTTACT |
| 2701 | GCTGATTTAA CAAAAATTTA ACGCGAATTA ATTCTGTGGA ATGTGTGTCA |
| | CGACTAAATT GTTTTTAAAT TGCGCTTAAT TAAGACACCT TACACACAGT |
| 2751 | GTTAGGGTGT GGAAAGTCCC CAGGCTCCCC AGCAGGCAGA AGTATGCAAA |
| | CAATCCCACA CCTTTCAGGG GTCCGAGGGG TCGTCCGTCT TCATACGTTT |
| 2801 | GCATGCATCT CAATTAGTCA GCAACCAGGT GTGGAAAGTC CCCAGGCTCC |
| | CGTACGTAGA GTTAATCAGT CGTTGGTCCA CACCTTTCAG GGGTCCGACG |
| 2851 | CCAGCAGGCA GAAGTATGCA AAGCATGCAT CTCAATTAGT CAGCAACCAT |
| | GGTCGTCCGT CTTCATACGT TTCGTACGTA GAGTTAATCA GTCGTTGGTA |
| 2901 | AGTCCCGCCC CTAACTCCGC CCATCCCGCC CCTAACTCCG CCCAGTTCCG |
| | TCAGGGCGGG GATTGAGGCG GGTAGGGCGG GGATTGAGGC GGGTCAAGGC |
| | NcoI |
| | ˜˜˜˜˜˜˜ |
| 2951 | CCCATTCTCC GCCCCATGGC TGACTAATTT TTTTTATTTA TGCAGAGGCC |
| | GGGTAAGAGG CGGGGTACCG ACTGATTAAA AAAAATAAAT ACGTCTCCGG |
| 3001 | GAGGCCGCCT CTGCCTCTGA GCTATTCCAG AAGTAGTGAG GAGGCTTTTT |
| | CTCCGGCGGA GACGGAGACT CGATAAGGTC TTCATCACTC CTCCGAAAAA |
| | XmaI |
| | ˜˜˜˜˜˜˜ |
| | SmaI |
| | ˜˜˜˜˜˜˜ |
| 3051 | TGGAGGCCTA GGCTTTTGCA AAAAGCTCCC GGGAGCTTCT ATATCCATTT |
| | ACCTCCGGAT CCGAAAACGT TTTTCGAGGG CCCTCGAACA TATAGGTAAA |
| | BclI |
| 3101 | TCGGATCTGA TCAAGAGACA GGATGAGGAT CGTTTCGCAT GATTGAACAA |
| | AGCCTAGACT AGTTCTCTGT CCTACTCCTA GCAAAGCGTA CTAACTTGTT |
| 3151 | GATGGATTGC ACGCAGGTTC TCCGGCCGCT TGGGTGGAGA GGCTATTCGG |
| | CTACCTAACG TGCGTCCAAG AGGCCGGCGA ACCCACCTCT CCGATAAGCC |
| 3201 | CTATGACTGG GCACAACAGA CAATCGGCTG CTCTGATGCC GCCGTGTTCC |
| | GATACTGACC CGTGTTGTCT GTTAGCCGAC GAGACTACGG CGGCACAAGG |
| 3251 | GGCTGTCAGC GCAGGGGCGC CCGGTTCTTT TTCTCAAGAC CGACCTGTCC |
| | CCGACAGTCG CGTCCCCGCG GGCCAAGAAA AACAGTTCTG GCTGGACAGG |
| | PstI MscI |
| | ˜˜˜˜˜˜˜ ˜˜˜˜ |
| 3301 | GGTGCCCTGA ATGAACTGCA GGACGAGGCA GCGCGGCTAT CGTGGCTGGC |
| | CCACGGGACT TACTTGACGT CCTGCTCCGT CGCGCCGATA GCACCGACCG |
| | MscI PvuII |
| | ˜˜ ˜˜˜˜˜˜ |
| 3351 | CACGACGGGC GTTCCTTGCG CAGCTGTGCT CGACGTTGTC ACTGAAGCGG |
| | GTGCTGCCCG CAAGGAACGC GTCGACACGA GCTGCAACAG TGACTTCGCC |
| 3401 | GAAGGGACTG GCTGCTATTG GGCGAAGTGC CGGGGCAGGA TCTCCTGTCA |
| | CTTCCCTGAC CGACGATAAC CCGCTTCACG GCCCCGTCCT AGAGGACAGT |
| 3451 | TCTCACCTTG CTCCTGCCGA GAAAGTATCC ATCATGGCTG ATGCAATGCG |
| | AGAGTGGAAC GAGGACGGCT CTTTCATAGG TAGTACCGAC TACGTTACGC |
| 3501 | GCGGCTGCAT ACGCTTGATC CGGCTACCTG CCCATTCGAC CACCAAGCGA |
| | CGCCGACQTA TGCGAACTAG GCCGATGGAC GGGTAAGCTG GTGGTTCGCT |
| 3551 | AACATCGCAT CGAGCGAGCA CGTACTCGGA TGGAAGCCGG TCTTGTCGAT |
| | TTGTAGCGTA GCTCGCTCGT GCATGAGCCT ACCTTCGGCC AGAACAGCTA |
| 3601 | CAGGATGATC TGGACGAAGA GCATCAGGGG CTCGCGCCAG CCGAACTGTT |
| | GTCCTACTAG ACCTGCTTCT CGTAGTCCCC GAGCGCGGTC GGCTTGACAA |
| | BssHII NcoI |
| | ˜˜˜˜˜˜ ˜˜ |
| 3651 | CGCCAGGCTC AAGGCGCGCA TGCCCGACGG CGAGGATCTC GTCGTGACCC |
| | GCGGTCCGAG TTCCGCGCGT ACGGGCTGCC GCTCCTAGAG CAGCACTGGG |
| | NcoI |
| | ˜˜˜˜ |
| 3701 | ATGGCGATGC CTGCTTGCCG AATATCATGG TGGAAAATGG CCGCTTTTCT |
| | TACCGCTACG GACGAACGGC TTATAGTACC ACCTTTTACC GGCGAAAAGA |
| 3751 | GGATTCATCG ACTGTGGCCG GCTGGGTGTG GCGGACCGCT ATCAGGACAT |
| | CCTAAGTAGC TCACACCGGC CGACCCACAC CGCCTGGCGA TAGTCCTGTA |
| 3801 | AGCGTTGGCT ACCCGTGATA TTGCTGAAGA GCTTGGCGGC GAATGGGCTG |
| | TCGCAACCGA TGGQCACTAT AACGACTTCT CCAACCGCCG CTTACCCGAC |
| 3851 | ACCGCTTCCT CGTGCTTTAC GGTATCGCCC CTCCCGATTC GCAGCGCATC |
| | TGGCGAAGGA GCACGAAATG CCATAGCGGC GAGGGCTAAG CGTCGCGTAG |
| | BstBI |
| | ˜˜˜ |
| 3901 | GCCTTCTATC GCCTTCTTGA CCAGTTCTTC TGAGCGGGAC TCTGGGGTTC |
| | CGGAAGATAG CGGAAGAACT GCTCAAGAAG ACTCGCCCTG AGACCCCAAG |
| | BstBI |
| | ˜˜˜ |
| 3951 | GAAATGACCG ACCAAGCGAC GCCCAACCTG CCATCACGAG ATTTCGATTC |
| | CTTTACTGGC TGGTTCGCTG CGGGTTGGAC GGTAGTGCTC TAAAGCTAAG |
| 4001 | CACCGCCGCC TTCTATGAAA GGTTGGGCTT CGGAATCGTT TTCCGGGACG |
| | GTGGCGGCGG AAGATACTTT CCAACCCGAA GCCTTAGCAA AAGGCCCTGC |
| 4051 | CCGGCTGGAT GATCCTCCAG CGCGGGGATC TCATGCTGGA GTTCTTCGCC |
| | GGCCGACCTA CTAGGAGGTC GCGCCCCTAG AGTACGACCT CAAGAAGCGG |
| 4101 | CACCCCAACT TGTTTATTGC AGCTTATAAT GGTTACAAAT AAAGCAATAG |
| | GTGGGGTTGA ACAAATAACG TCGAATATTA CCAATGTTTA TTTCGTTATC |
| 4151 | CATCACAAAT TTCACAAATA AAGCATTTTT TTCACTGCAT TCTAGTTGTG |
| | GTAGTGTTTA AAGTGTTTAT TTCGTAAAAA AAGTGACGTA AGATCAACAC |
| | SalI |
| | ˜˜˜˜˜˜ |
| 4201 | GTTTGTCCAA ACTCATCAAT GTATCTTATC ATGTCTGTAT ACCGTCGACC |
| | CAAACAGGTT TGAGTAGTTA CATAGAATAG TACAGACATA TGGCAGCTGG |
| 4251 | TCTAGCTAGA GCTTGGCGTA ATCATGGTCA TAGCTGTTTC CTGTGTGAAA |
| | AGATCGATCT CGAACCGCAT TAGTACCAGT ATCGACAAAG GACACACTTT |
| 4301 | TTGTTATCCG CTCACAATTC CACACAACAT ACGAGCCGGA AGCATAAAGT |
| | AACAATAGGC GAGTGTTAAG GTGTGTTGTA TGCTCGGCCT TCGTATTTCA |
| 4351 | GTAAAGCCTG GGGTGCCTAA TGAGTGAGCT AACTCACATT AATTGCGTTG |
| | CATTTCGGAC CCCACGGATT ACTCACTCGA TTGAGTGTAA TTAACGCAAC |
| | PvuII |
| | ˜˜˜˜˜˜ |
| 4401 | CGCTCACTGC CCGCTTTCCA GTCGGGAAAC CTGTCGTGCC AGCTGCATTA |
| | GCGAGTGACG GGCGAAAGGT CAGCCCTTTG GACAGCACGG TCGACGTAAT |
| 4451 | ATGAATCGGC CAACGCGCGG GGAGAGGCGG TTTGCGTATT GGGCGCTCTT |
| | TACTTAGCCG GTTGCGCGCC CCTCTCCGCC AAACGCATAA CCCGCGAGAA |
| 4501 | CCGCTTCCTC GCTCACTGAC TCGCTGCGCT CGGTCGTTCG GCTGCGGCGA |
| | GGCGAAGGAG CGAGTGACTG AGCGACGCGA GCCAGCAAGC CGACGCCGCT |
| 4551 | GCGGTATCAG CTCACTCAAA GGCGGTAATA CGGTTATCCA CAGAATCAGG |
| | CGCCATAGTC GAGTGAGTTT CCGCCATTAT GCCAATAGGT GTCTTAGTCC |
| 4601 | GGATAACGCA GGAAAGAACA TGTGAGCAAA AGGCCAGCAA AAGGCCAGGA |
| | CCTATTGCGT CCTTTCTTGT ACACTCGTTT TCCGGTCGTT TTCCGGTCCT |
| 4651 | ACCGTAAAAA GGCCGCGTTG CTGGCGTTTT TCCATAGGCT CCGCCCCCCT |
| | TGGCATTTTT CCGGCGCAAC GACCGCAAAA AGGTATCCGA GGCGGGGGGA |
| 4701 | GACGAGCATC ACAAAAATCG ACGCTCAAGT CAGAGGTGGC GAAACCCGAC |
| | CTGCTCGTAG TGTTTTTAGC TGCGAGTTCA GTCTCCACCG CTTTGGGCTG |
| 4751 | AGGACTATAA AGATACCAGG CGTTTCCCCC TGGAAGCTCC CTCGTGCGCT |
| | TCCTGATATT TCTATGGTCC GCAAAGGGGG ACCTTCGAGG GAGCACGCGA |
| 4801 | CTCCTGTTCC GACCCTGCCG CTTACCGGAT ACCTGTCCGC CTTTCTCCCT |
| | GAGGACAAGG CTGGGACGGC GAATGGCCTA TGGACAGGCG GAAAGAGGCA |
| 4851 | TCGGGAAGCG TGGCGCTTTC TCATAGCTCA CGCTGTAGGT ATCTCAGTTC |
| | AGCCCTTCGC ACCGCGAAAG AGTATCGAGT GCGACATCCA TAGAGTCAAG |
| 4901 | GGTGTAGGTC GTTCGCTCCA AGCTGGGCTG TGTGCACGAA CCCCCCGTTC |
| | CCACATCCAG CAAGCGAGGT TCOACCCGAC ACACGTGCTT GGGGGGCAAG |
| 4951 | AGCCCGACCG CTGCGCCTTA TCCGGTAACT ATCGTCTTGA GTCCAACCCG |
| | TCGGGCTGGC GACGCGGAAT AGGCCATTGA TAGCAGAACT CAGGTTGGGC |
| 5001 | GTAAGACACG ACTTATCGCC ACTGGCAGCA GCCACTGGTA ACAGGATTAG |
| | CATTCTGTGC TGAATAGCGG TGACCGTCGT CGGTGACCAT TGTCCTAATC |
| 5051 | CAGAGCGAGG TATGTAGGCG GTGCTACAGA GTTCTTGAAG TGGTGGCCTA |
| | GTCTCGCTCC ATACATCCGC CACGATGTCT CAAGAACTTC ACCACCGGAT |
| 5101 | ACTACGGCTA CACTAGAAGA ACAGTATTTG GTATCTGCGC TCTGCTGAAG |
| | TGATGCCGAT GTGATCTTCT TGTCATAAAC CATAGACGCG AGACGACTTC |
| 5151 | CCAGTTACCT TCGGAAAAAG AGTTGGTAGC TCTTGATCCG GCAAACAAAC |
| | GGTCAATGGA AGCCTTTTTC TCAACCATCG AGAACTAGGC CGTTTGTTTG |
| 5201 | CACCGCTGGT AGCGGTTTTT TTGTTTGCAA GCAGCAGATT ACGCGCAGAA |
| | GTGGCGACCA TCGCCAAAAA AACAAACGTT CGTCGTCTAA TGCGCGTCTT |
| 5251 | AAAAAGGATC TCAAGAAGAT CCTTTGATCT TTTCTACGGG GTCTGACGCT |
| | TTTTTCCTAG AGTTCTTCTA GGAAACTAGA AAAGATGCCC CAGACTGCGA |
| 5301 | CAGTGGAACG AAAACTCACG TTAAGGGATT TTGGTCATGA GATTATCAAA |
| | GTCACCTTGC TTTTGAGTGC AATTCCCTAA AACCAGTACT CTAATAGTTT |
| 5351 | AAGGATCTTC ACCTAGATCC TTTTAAATTA AAAATGAAGT TTTAAATCAA |
| | TTCCTAGAAG TGGATCTAGG AAAATTTAAT TTTTACTTCA AAATTTAGTT |
| 5401 | TCTAAAGTAT ATATGAGTAA ACTTGGTCTG ACAGTTACCA ATGCTTAATC |
| | AGATTTCATA TATACTCATT TGAACCAGAC TGTCAATGGT TACGAATTAG |
| 5451 | AGTGAGGCAC CTATCTCAGC GATCTGTCTA TTTCGTTCAT CCATAGTTGC |
| | TCACTCCGTG GATAGAGTCG CTAGACAGAT AAAGCAAGTA GGTATCAACG |
| 5501 | CTGACTCCCC GTCGTGTAGA TAACTACGAT ACGGGAGGGC TTACCATCTG |
| | GACTGAGGGG CAGCACATCT ATTGATGCTA TGCCCTCCCG AATGGTAGAC |
| 5551 | GCCCCAGTGC TGCAATGATA CCGCGAGACC CACGCTCACC GGCTCCAGAT |
| | CGGGGTCACG ACGTTACTAT GGCGCTCTGG GTGCGAGTGG CCGAGGTCTA |
| 5601 | TTATCAGCAA TAAACCAGCC AGCCGGAAGG GCCGAGCGCA GAAGTGGTCC |
| | AATAGTCGTT ATTTGGTCGG TCGGCCTTCC CGGCTCGCGT CTTCACCAGG |
| 5651 | TGCAACTTTA TCCGCCTCCA TCCAGTCTAT TAATTGTTGC CGGGAAGCTA |
| | ACGTTGAAAT AGGCGGAGGT AGGTCAGATA ATTAACAACG GCCCTTCGAT |
| 5701 | GAGTAAGTAG TTCGCCAGTT AATAGTTTGC GCAACGTTGT TGCCATTGCT |
| | CTCATTCATC AAGCGGTCAA TTATCAAACG CGTTGCAACA ACGGTAACGA |
| 5751 | ACAGGCATCG TGGTGTCACG CTCGTCGTTT GGTATGGCTT CATTCAGCTC |
| | TGTCCGTAGC ACCACAGTGC GAGCAGCAAA CCATACCGAA GTAAGTCGAG |
| 5801 | CGGTTCCCAA CGATCAAGGC GAGTTACATG ATCCCCCATG TTGTGCAAAA |
| | GCCAAGGGTT GCTAGTTCCG CTCAATGTAC TAGGGGGTAC AACACGTTTT |
| | PvuI |
| | ˜˜˜˜˜˜ |
| 5851 | AAGCGGTTAG CTCCTTCGGT CCTCCGATCG TTGTCAGAAG TAAGTTGGCC |
| | TTCGCCAATC GAGGAAGCCA GGAGGCTAGC AACAGTCTTC ATTCAACCGG |
| 5901 | GCAGTGTTAT CACTCATGGT TATGGCAGCA CTGCATAATT CTCTTACTGT |
| | CGTCACAATA GTGAGTACCA ATACCGTCGT GACGTATTAA GAGAATGACA |
| 5951 | CATGCCATCC GTAAGATGCT TTTCTGTGAC TGGTGAGTAC TCAACCAAGT |
| | GTACGGTAGG CATTCTACGA AAAGACACTG ACCACTCATG AGTTGGTTCA |
| 6001 | CATTCTGAGA ATAGTGTATG CGGCGACCGA GTTGCTCTTG CCCGGCGTCA |
| | GTAAGACTCT TATCACATAC GCCGCTGGCT CAACGAGAAC GGGCCGCAGT |
| 6051 | ATACGGGATA ATACCGCGCC ACATAGCAGA ACTTTAAAAG TGCTCATCAT |
| | TATGCCCTAT TATGGCGCGG TGTATCGTCT TGAAATTTTC ACGAGTAGTA |
| 6101 | TGGAAAACGT TCTTCGGGGC GAAAACTCTC AAGGATCTTA CCGCTGTTGA |
| | ACCTTTTGCA AGAAGCCCCG CTTTTGAGAG TTCCTAGAAT GGCGACAACT |
| 6151 | GATCCAGTTC GATGTAACCC ACTCGTGCAC CCAACTGATC TTCAGCATCT |
| | CTAGGTCAAG CTACATTGGG TGAGCACGTG GGTTGACTAG AAGTCGTAGA |
| 6201 | TTTACTTTCA CCAGCGTTTC TGGGTGAGCA AAAACAGGAA GGCAAAATGC |
| | AAATGAAAGT GGTCGCAAAG ACCCACTCGT TTTTGTCCTT CCGTTTTACG |
| 6251 | CGCAAAAAAG GGAATAAGGG CGACACGGAA ATGTTGAATA CTCATACTCT |
| | GCGTTTTTTC CCTTATTCCC GCTGTGCCTT TACAACTTAT GAGTATGAGA |
| 6301 | TCCTTTTTCA ATATTATTGA AGCATTTATC AGGGTTATTG TCTCATGAGC |
| | AGGAAAAAGT TATAATAACT TCGTAAATAG TCCCAATAAC AGAGTACTCG |
| 6351 | GOATACATAT TTGAATGTAT TTAGAAAAAT AAACAAATAG GGGTTCCGCG |
| | CCTATGTATA AACTTACATA AATCTTTTTA TTTGTTTATC CCCAAGGCGC |
| | SalI |
| | ˜˜˜˜ |
| 6401 | CACATTTCCC CGAAAAGTGC CACCTGACGT C |
| | GTGTAAAGGG GCTTTTCACG GTGGACTGCA G |

### REFERENCE LIST

Alewijnse A E, Timmerman H, Jacobs E H, Smit M J, Roovers E, Cotecchia S and Leurs R (2000) The Effect of Mutations in the DRY Motif on the Constitutive Activity and Structural Instability of the Histamine H₂ Receptor. Mol Pharmacol 57: pp 890-898.

An S, Zheng Y and Bleu T (2000) Sphingosine 1-Phosphate-Induced Cell Proliferation, Survival, and Related Signaling Events Mediated by G Protein-Coupled Receptors Edg3 and Edg5. J Biol Chem 275: pp 288-296.

Botstein D, Chervitz S A and Cherry J M (1997) Yeast As a Model Organism. Science 277: pp 1259-1260.

Brown A J, Dyos S L, Whiteway M S, White J H, Watson M A, Marzioch M, Clare J J, Cousens D J, Paddon C, Plumpton C, Romanos M A and Dowell S J (2000) Functional Coupling of Mammalian Receptors to the Yeast Mating Pathway Using Novel Yeast/Mammalian G Protein α-Subunit Chimeras. Yeast 16: pp 11-22.

Chambers J K, Macdonald L E, Sarau H M, Ames R S, Freeman K, Foley J J, Zhu Y, McLaughlin M M, Murdock P, McMillan L, Trill J, Swift A, Aiyar N, Taylor P, Vawter L, Naheed S, Szekeres P, Hervieu G, Scott C, Watson J M, Murphy A J, Duzic E, Klein C, Bergsma D J, Wilson S and Livi G P (2000) A G Protein-Coupled Receptor for UDP-Glucose. J Biol Chem 275: pp 10767-10771.

Chen G, Way J, Armour S, Watson C, Queen K, Jayawickreme C K, Chen W J and Kenakin T (2000) Use of Constitutive G Protein-Coupled Receptor Activity for Drug Discovery. Mol Pharmacol 57: pp 125-134.

Daeffler L and Landry Y (2000) Inverse Agonism at Heptahelical Receptors: Concept, Experimental Approach and Therapeutic Potential. Fundam Clin Pharmacol 14: pp 73-87.

Dhanasekaran N, Heasley L E and Johnson G L (1995) G Protein-Coupled Receptor Systems Involved in Cell Growth and Oncogenesis. Endocr Rev 16: pp 259-270.

Duprez L, Parma J, Costagliola S, Hermans J, Van Sande J, Dumont J E and Vassart G (1997) Constitutive Activation of the TSH Receptor by Spontaneous Mutations Affecting the N-Terminal Extracellular Domain. FEBS Lett 409: pp 469-474.

Egan C T, Herrick-Davis K and Teitler M (1998) Creation of a Constitutively Activated State of the 5-Hydroxytryptamine2A Receptor by Site-Directed Mutagenesis: Inverse Agonist Activity of Antipsychotic Drugs. J Pharmacol Exp Ther 286: pp 85-90.

Erlenbach I, Kostenis E, Schmidt C, Serradeil-Le Gal C, Raufaste D, Dumont M E, Pausch M H and Wess J (2001) Single Amino Acid Substitutions and Deletions That Alter the G Protein Coupling Properties of the V2 Vasopressin Receptor Identified In Yeast by Receptor Random Mutagenesis. J Biol Chem pp M103203200.

Hadcock J R and Pausch M (1999) Ligand screening of G protein-coupled receptors in yeast, in G Protein-Coupled Receptors (Haga T and Berstein G eds) pp 49-69, CRC Press LLC, Boca Raton, Fla.

Hla T (2001) Sphingosine 1-Phosphate Receptors. Prostaglandins & Other Lipid Mediators 64: pp 135-142.

Ito H, Fukuda Y, Murata K and Kimura A (1983) Transformation of Intact Yeast Cells Treated With Alkali Cations. J Bacteriol 153: pp 163-168.

Jensen A A, Spalding T A, Burstein E S, Sheppard P O, O'Hara P J, Brann M R, Krogsgaard-Larsen P and Bräuner-Osborne H (2000) Functional Importance of the Ala¹¹⁶-Pro¹³⁶ Region in the Calcium-Sensing Receptor. J Biol Chem 275: pp 29547-29555.

Konopka J B, Margarit S M and Dube P (1996) Mutation of Pro-258 in Transmembrane Domain 6 Constitutively Activates the G Protein-Coupled α-Factor Receptor. Proc Natl Acad Sci USA 93: pp 6764-6769.

Lefkowitz R J, Cotecchia S, Samama P and Costa T (1993) Constitutive Activity of Receptors Coupled to Guanine Nucleotide Regulatory Proteins. Trends Pharmacol Sci 14: pp 303-307.

Ma H, Kunes S, Schatz P J and Botstein D (1987) Plasmid Construction by Homologous Recombination in Yeast. Gene 58: pp 201-216.

MacEwan D J and Milligan G (1996) Inverse Agonist-Induced Up-Regulation of the Human B₂-Adrenoceptor in Transfected Neuroblastoma X Glioma Hybrid Cells. Mol Pharmacol 50: pp 1479-1486.

Oldenburg K R, Vo K T, Michaelis S and Paddon C (1997) Recombination-Mediated PCR-Directed Plasmid Construction in Vivo in Yeast. Nucleic Acids Res 25: pp 451-452.

Parnot C, Bardin S, Miserey-Lenkei S, Guedin D, Corvol P and Clauser E (2000) Systematic Identification of Mutations That Constitutively Activate the Angiotensin II Type 1A Receptor by Screening a Randomly Mutated CDNA Library With an Original Pharmacological Bioassay. Proc Natl Acad Sci USA 97: pp 7615-7620.

Price L A, Kajkowski E M, Hadcock J R, Ozenberger B A and Pausch M H (1995) Functional Coupling of a Mammalian Somatostatin Receptor to the Yeast Pheromone Response Pathway. Mol Cell Biol 15: pp 6188-6195.

Rao V R and Oprian D D (1996) Activating Mutations of Rhodopsin and Other G Protein-Coupled Receptors. Annu Rev Biophys Biomol Struct 25: pp 287-314.

Robinson P R, Cohen G B, Zhukovsky E A and Oprian D D (1992) Constitutively Active Mutants of Rhodopsin. Neuron 9: pp 719-725.

Samama P, Bond R A, Rockman H A, Milano C A and Lefkowitz R J (1997) Ligand-Induced Overexpression of a Constitutively Active B₂-Adrenergic Receptor: Pharmacological Creation of a Phenotype in Transgenic Mice. Proc Natl Acad Sci USA 94: pp 137-141.

Sommers C M and Dumont M E (1997) Genetic Interactions Among the Transmembrane Segments of the G Protein Coupled Receptor Encoded by the Yeast STE2 Gene. J Mol Biol 266: pp 559-575.

Sommers C M, Martin N P, Akal-Strader A, Becker J M, Naider F and Dumont M E (2000) A Limited Spectrum of Mutations Causes Constitutive Activation of the Yeast Alpha-Factor Receptor [In Process Citation]. Biochemistry 39: pp 6898-6909.

Spiegel A M (1996) Mutations in G Proteins and G Protein-Coupled Receptors in Endocrine Disease. J Clin Endocrinol Metab 81: pp 2434-2442.

Svetlov V and Cooper T G (1998) Efficient PCR-Based Random Mutagenesis of Sub-Genic (100 Bp) DNA Fragments. Yeast 14: pp 89-91.

Tate C G and Grisshammer R (1996) Heterologous Expression of G-Protein-Coupled Receptors. Trends Biotechnol 14: pp 426-430.

Tsao P, Cao T and von Zastrow M (2001) Role of Endocytosis in Mediating Downregulation of G-Protein-Coupled Receptors. Trends Pharmacol Sci 22: pp 91-96.

Varma D R, Shen H, Deng X F, Peri K G, Chemtob S and Mulay S (1999) Inverse Agonist Activities of B-Adrenoceptor Antagonists in Rat Myocardium. Br J Pharmacol 127: pp 895-902.

Zwick E, Hackel P O, Prenzel N and Ullrich A (1999) The EGF Receptor As Central Transducer of Heterologous Signalling Systems. Trends Pharmacol Sci 20: pp 408-412.

1. Method of identifying protein CAMs (constitutively active mutants) wherein
a) a library of mutated sequences of a protein is generated,
b) yeast cells are transformed with such library, and
c) the respective protein CAM is identified.

2. Method of identifying protein CAMs (constitutively active mutants) wherein
d) a library of mutated sequences of a protein is generated,
e) yeast cells are co-transformed with the library and a linearized expression vector,
f) the transformed yeast cells are selected for the repair of the plasmid, and
g) protein CAMs are identified by determining the activity of the respective protein mutant.

3. Method as claimed in claim 1 or 2, wherein the protein is a GPCR (G-Protein coupled receptor), an ion-channel or an enzyme.

4. Method as claimed in claim 3, wherein the enzyme is a kinase.

5. Method as claimed in one of the foregoing claims, wherein the protein is a mammalian protein.

6. Use of the method as claimed in claims 1 to 5, for identifying agonists or inverse agonists.


SEQUENCE LISTING (4 sequences)

SEQ ID NO: 1 - DNA, 22 bp, Artificial
  Synthetic nucleotide primer
atgggcagcttgtactcggagt

SEQ ID NO: 2 - DNA, 21 bp, Artificial
  Synthetic Nucleotide Primer
tcagaacaccgtgttgccctc

SEQ ID NO: 3 - DNA, 6831 bp, Artificial
  Nucleotide Sequence of p416 GPD-Edge5
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccggccgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgactcgaggtcgactcagaacaccgtgttgccctccagaaacgtgggtgacgtgggcatgtgcatgcccctctccagggagctggagctgcggagtggcaggaggtggtggcccggggtcccgccccgcctccgtccttgcacccccacccccggccgccagcactgcagcggccgaagcacctcccgccgcaggtcccggctgcgccacgtgtagatgacggggttgagcagggaattcagggtggagacggcgaaaaagtagtgggctttgtagaggatcgggcaggagtggacgggacaggcatagtccagaaggaggatgctgaaggcgggcagccagcagacgataaagacgcctagcacgatggtgaccgtcttgagcagggctagcgtctgcggggcggccatgtcagcgtggcttgagcggaccacgcagtagatgcgcacgtacagggccacgatggccaacaggatgatggagaagatggtcaccacgcacagcacataatgcttggcgtagagaggcaggacagtggagcaggcctcgaggtggcccaggcagttccagccaaggatgggcaggccaccgaggaccagcgagatgagccacgaggccccgatgagcagaagcatgcggcagctcttgtcgctgccatacagcttgaccttggcaatggccacgtggcgctcaatggcgatggccaggaggctgaagacagaggccgagagcgtgatgaaggcagagccctcccgggcaaaccactgcacaggcgtcagcctcagcgtgacagagccagagagcaaggtattggctacgaaggccacgcctgccagtagatcggaggcggccaggttgcccagaaacaggtacattgccgagtggaacttgctgtttcgggccaccgcaatgagcgctagcaggttttccaccacaatggcgcaacagaggatgacgatgaaggccgaggccacctggcgggaggtcgtctcctgcgtttccagcgtctccttggtataattatagtgttcctggaccttgttggggttcaggtactccgagtacaagctgcccattttatcgataagcttgatatcgaattcctgcagcccggctagttctagaatccgtcgaaactaagttctggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgcccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactgagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 4 - DNA, 6431 bp, Artificial
  Nucleotide Sequence of pcDNA3.1(+) - Edge 5
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatcgataaaatgggcagcttgtactcggagtacctgaaccccaacaaggtccaggaacactataattataccaaggagacgctggaaacgcaggagacgacctcccgccaggtggcctcggccttcatcgtcatcctctgttgcgccattgtggtggaaaaccttctggtgctcattgcggtggcccgaaacagcaagttccactcggcaatgtacctgtttctgggcaacctggccgcctccgatctactggcaggcgtggccttcgtagccaataccttgctctctggctctgtcacgctgaggctgacgcctgtgcagtggtttgcccgggagggctctgccttcatcacgctctcggcctctgtcttcagcctcctggccatcgccattgagcgccacgtggccattgccaaggtcaagctgtatggcagcgacaagagctgccgcatgcttctgctcatcggggcctcgtggctcatctcgctggtcctcggtggcctgcccatccttggctggaactgcctgggccacctcgaggcctgctccactgtcctgcctctctacgccaagcattatgtgctgtgcgtggtgaccatcttctccatcatcctgttggccatcgtggccctgtacgtgcgcatctactgcgtggtccgctcaagccacgctgacatggccgccccgcagacgctagccctgctcaagacggtcaccatcgtgctaggcgtctttatcgtctgctggctgcccgccttcagcatcctccttctggactatgcctgtcccgtccactcctgcccgatcctctacaaagcccactactttttcgccgtctccaccctgaattccctgctcaaccccgtcatctacacgtggcgcagccgggacctgcggcgggaggtgcttcggccgctgcagtgctggcggccgggggtgggggtgcaaggacggaggcggggcgggaccccgggccaccacctcctgccactccgcagctccagctccctggagaggggcatgcacatgcccacgtcacccacgtttctggagggcaacacggtgttctgagtcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc